(19) United States
(12) Patent Application Publication
(10) Pub. No.: US 2026/0216002 A1
(43) Pub. Date: Jul. 30, 2026

(54) DRUG MIXING SYSTEM

(71) Applicant: AspiAir GmbH, Gemünden (DE)

(72) Inventors: Axel Hans-Otto Fischer, Gilserberg (DE); Tobias Kolb, Neuried (DE)

(21) Appl. No.: 19/143,394

(22) PCT Filed: Dec. 27, 2023
(86) PCT No.: PCT/EP2023/087840

§ 371 Date: Jun. 26, 2025

(30) Foreign Application Priority Data
Dec. 30, 2022 (EP) 22217382.5

(51) Int. Cl. A61J 1/20; A61J 1/16; A61M 15/00

(52) U.S. Cl. CPC A61J 1/2096; A61J 1/16; A61J 1/2013; A61J 1/2055; A61J 1/2062; A61M 15/0085

(57) ABSTRACT

The invention provides a drug mixing system (10) for mixing of a drug formulation (110a) stored in a vial (110), such as a powder for reconstitution or a drug concentrate, with a liquid stored in a separate reservoir (120), and then transferring the dissolved or diluted drug formulation (110b) to either a target site or into a receiving device (200), such as a nebulizer (201), for subsequent administration. The drug mixing system (10) aims at simplifying and improving both the mixing-and the transferring steps, inter alia, by reducing the number of steps required as well as limiting the risk for contamination and/or spillage of the dissolved or diluted drug formulation (110b). For this purpose, the drug mixing system (10) uses an overpressure generated upon transferring the liquid from the reservoir (120) into the vial (110) with the help of a piston (125) and then locking the piston (125) in place. The release of said overpressure by expelling the dissolved or diluted drug formulation (110b) from the vial (110) through an outlet port (130) of the drug mixing system (10) and into the receiving device (200) is preferably actuated automatically upon connecting the drug mixing system (10) and receiving device (200).

The invention relates to a drug mixing system, in particular, to a drug mixing system facilitating and simplifying the transfer of drug formulations prepared freshly short before use (e.g., dry powder formulations of drug intended for reconstitution) to a target administration site and/or to a receiving device, such as a nebulizer.

### BACKGROUND OF THE INVENTION

Drugs intended for administration to patients are often provided either as dry powders (e.g., lyophilisates) or in liquid form (e.g., a liquid concentrate or solubilisate). Prior to administration, these drugs in powdered or liquid form typically require reconstitution, or dilution, with a pharmaceutically acceptable liquid, or medium (for instance, a solvent such as water for injection purposes), said liquid, or medium, being provided in a separate vessel than the drug. Reconstitution of the drug for administration to the patient should be performed with as little loss as possible and by transferring the pharmaceutically acceptable liquid under sterile conditions to the drug and to mix the drug with the pharmaceutically acceptable liquid.

Many prior art systems require a multitude of handling steps in order to prepare the system for mixing and injection prior to use, such as breaking an ampoule containing the injectable medium or piercing a rubber closure on a vial or bottle filled with the injectable medium using a needle, aspiration of the injectable medium into a syringe, transfer of the aspired medium to a vial containing a drug formulation and often several further steps to mix the drug with the injectable medium and in most cases dissolve it therein (e.g., create physical agitation by either shaking the vial and/or by transferring the drug-medium blend back and forth between the vial and the syringe). Thereafter, if the resulting drug formulation is intended for injection into a patient (rather than e.g., some test device), it is then often required to change the needle attached to the syringe prior to injection, since the needle diameters used for convenient handling and mixing are often larger than those allowing for easy and painfree injection.

Moreover, in prior art systems where there is no direct connection between the drug containing vial and the medium containing syringe, any transfer of either the injectable liquid and/or the drug formulation resulting from mixing the drug with said injectable liquid can entrain air into the system, and in consequence air-borne microbes.

Furthermore, many users, or patients, are hesitant to e.g., open glass ampoules and/or to prepare injectable drug formulations by themselves handling multiple needles. Hence, oftentimes either compliance to medication requiring the above ‘prepare right before use’ procedures is low, and/or the preparation steps outlined above have to be carried out by medically trained staff or caretakers. In summary, the prior art systems, and in particular the multiple steps required with them, are not only susceptible to contamination but also not very user-friendly.

Attempts to overcome some of the above challenges have been made, especially for drugs intended for subsequent injection using needles and syringes. For instance, US20130046270A1 (herein ‘US 270’) or W02021094548A1 (herein ‘WO 548’) describe mixing and injection systems comprising adapters with openings adapted to hold two containers; a ‘drug container’ housing a drug formulation (such as a glass vial), and a ‘liquid container’ housing a liquid for dissolving or diluting the drug formulation. The adapters in both US 270 or WO 548 comprise two movably engageable subunits so as to allow for an activation step of, or with, the adapter which results in the initially closed drug container (optionally both the drug container and the liquid container) to be pierced open; namely by pressing the two subunits of the adapter together or pushing them towards each other. Upon activation, the liquid can then be transferred from the liquid container into the drug container without the drug formulation and/or the liquid for its dissolution or dilution getting in contact with a user's hands, and further without the user being exposed to any open needle tips during this transfer step.

US 270 only offers only a unidirectional liquid flow from a vial-type liquid container, or ‘liquid vial’, to a vial-type drug container, or ‘drug vial’, driven by the vacuum oftentimes present in such drug vials (e.g., vials of lyophilized drug formulations); in other words, once the closure of the drug vial of US 270 is pierced open, the underpressure in said drug vial draws in the liquid from the liquid vial. In contrast, WO 548 uses a syringe instead of US 270′s liquid vial as the liquid container and thereby offers the option of a back-and forth flow between the drug container and the liquid container which can be advantageous, for instance, for drugs with solubility issues (e.g., poorly soluble drugs) which may then benefit from a more thorough mixing process. Moreover, unlike US 270 which requires the dissolved drug composition to be drawn into a separate injection syringe (i.e., a third container attached, or attachable, to their adapter which is then detached from the adapter for injection), WO 548 already uses a syringe as the liquid container and thus allows for not only the back-and forth mixing mentioned above but also for (i) a more compact, less material-consumptive mixing and injection system and (ii) for an easier injection by simply attaching an injection needle to the adapter's outlet port and without having to detach the syringe from the adapter. In other words, the syringe in the mixing and injection system described in WO 548 doubles as both the liquid container housing the liquid for dissolving or diluting the drug formulation and as the actual injection syringe.

However, both systems of US 270 or WO 548 are clearly tailored towards preparing drug formulations for injection by either dissolving or diluting them with a separately stored liquid and then inject them into a recipient's body (e.g., into a human patient) with the help of injection needles. Neither US 270 nor WO 548 address any other means of applying, or using, the freshly dissolved/diluted drug formulation, or how users of their systems could be supported further if the application mode is not injection; for instance, if the freshly dissolved/diluted drug formulation has to be transferred, preferably without contamination or product losses, to a different site of action or into receiving devices other than syringes.

Thus, further challenges can occur here for users, especially for users experiencing impaired mobility and/or vision impairments.

Similar considerations apply to US20040030285A1 (herein ‘US 285’) which also describes a drug delivery device for mixing and delivering a drug by injection, in particular a drug delivery system that uses an overpressure for injecting a dissolved drug formulation into human skin, said overpressure being generated in a drug vial upon forcing diluent (e.g., 1 mL thereof) from a diluent cartridge into the drug vial, thereby compressing the ambient air therein. However, in order to store the overpressure, the user of the device needs to manually hold down the inserted liquid cartridge while at the same time also manually pressing a release mechanism to move the injection needle into the skin. Moreover, in order to prevent injection of the compressed air into the skin, the outlet opening-in US 285 an injection needle-must be provided at an angle of approx. 90° to the longitudinal central axis of the drug vial, and the dissolved drug formulation delivered to this outlet opening via tortuous flow channels/tubes. This set-up increases both the dead-volume in which drug formulation is lost and can cause issues for drug formulations that are not solutions, e.g., emulsions or suspensions. Moreover, US 285 teaches that the pressure is only adjustable by changes to the liquid cartridge (e.g., less liquid) which means that there is limited flexibility with standardized, prefilled cartridges.

It is thus an object of the present invention to provide devices that avoid these drawbacks of the prior art; for instance, by providing drug mixing systems, or devices, that are easy to use (e.g., intuitive and single-handedly as far as possible), offer low susceptibility to tampering or even being tamper-proof, require few components (i.e., low material consumption), which further offer reduced drug formulation losses as well as improved flexibility when working with standardized, prefilled drug and/or liquid containers, and which reduce the contamination risk during the dissolution or dilution, preferably under aseptic conditions, of a drug formulation prior to administration to a minimum.

### SUMMARY OF THE INVENTION

In a first aspect, the invention relates to a drug mixing system (10) for dissolving or diluting a drug formulation (110a) stored in a vial (110) prior to use, the drug mixing system (10) comprising an adapter (100) adapted to hold the vial (110) housing the drug formulation (110a), and a reservoir (120) for housing a liquid (120a) for dissolution or dilution of the drug formulation (110a), the reservoir (120) comprising a housing (124) and a piston (125) sealingly movable within the housing, wherein the adapter (100) comprises a vial mounting opening (111) adapted to hold the vial (110), a reservoir mounting opening (121) adapted to hold the reservoir (120), an outlet port (130), an internal adapter channel (140) fluidly connecting the vial-and reservoir mounting openings (111, 121), and the outlet port (130), and a liquid-retaining means (145) adapted to block at least a flow of dissolved or diluted drug formulation (110b) through the outlet port (130), characterized in that the adapter (100) is equipped with a piston locking means (126) adapted to lock the position of the piston (125) once a predetermined volume of the liquid has been transferred from the reservoir (120) into the vial (110) by means of pressing the piston (125) into the housing (124), thereby creating and storing an overpressure, and in that the drug mixing system (10) is adapted to expel, or transfer, the dissolved or diluted drug formulation (110b) from the vial (110) through the outlet port (130) upon actuation of a liquid-releasing means (155, 255) using the overpressure generated by pressing the piston (125) into the housing (124).

In a second aspect, the present invention relates to an adapter (100) for the drug mixing system (10) according to the first aspect of the invention.

In a third aspect, the present invention relates to a method of preparing a dissolved or diluted drug formulation (110b), optionally a drug solution, from a drug formulation (110a) stored in a vial (110), using the drug mixing system (10) according to the first aspect of the invention; or, more specifically, a method comprising the following subsequent steps:
a) providing a drug mixing system (10) according to the first aspect of the invention;
b) with the liquid-retaining means (145) blocking at least the flow of dissolved or diluted drug formulation (110b) through the outlet port (130), transferring a predetermined volume of the liquid from the reservoir (120) into the vial (110) by means of pressing the piston (125) into the housing (124), thereby creating an overpressure and a mixture of the drug formulation (110a) and the liquid;
c) locking the piston (125) in place with the piston locking means (126) once the predetermined volume of the liquid has been transferred from the reservoir (120) into the vial (110), thereby storing the overpressure created in step b;
d) allowing the mixture of the drug formulation (110a) and the liquid obtained from steps b) and c) to get dissolved or diluted to the desired extent; optionally aiding this dissolution or dilution process by shaking the drug mixing system (10);
e) actuating the liquid-releasing means (155, 255), thereby expelling, or transferring, the dissolved or diluted drug formulation (110b) from the vial (110) through the outlet port (130) using the overpressure generated and stored in steps b) and c) by pressing the piston (125) into the housing (124) and locking the piston (125) in place.

Further objects, aspects, useful embodiments, applications, beneficial effects, and advantages of the invention will become apparent on the basis of the description of the invention, the examples, figures and claims below.

### DESCRIPTION OF THE FIGURES

The following examples and figures serve to illustrate the invention in exemplary embodiments; however, they should not be understood as restricting the scope of the invention. Any reference signs used in the claims or throughout the description should not be construed as a limitation to the embodiments represented in any of the figures. Further details on the figures follow in the detailed description section below.

It should further be understood that the figures provided herein represent depictions of the devices and components discussed herein which been simplified to a degree that allows discussion of said devices and components with respect to their functions, effects, and interrelations, yet without necessarily depicting each and every minor detail that may additionally be present in the devices or systems of the present invention. The description may thus contain details not all of which are necessarily visible in the drawings.

FIGS. 1A-1E depict cross-sectional front views of an exemplary embodiment of a drug mixing system (10) according to the first aspect of the invention in its normal operational, upright position, more specifically a seal-type drug mixing system (10a). The vial (110) and the reservoir (120) are arranged side-by-side in the adapter (100/100a) with their respective longitudinal central axes ‘A’ and ‘B’ being arranged in parallel (see FIG. 1E). The system's adapter (100/100a), comprising two adapter subunits (101, 102), is shown in both its resting position (FIG. 1A; vial (110) and reservoir (120) sealed; subunits slightly apart), and in its activated position (FIG. 1B; vial (110) and reservoir (120) pierced; subunits moved towards each other). FIG. 1C shows the system while the piston (125) is being pressed into the housing (124) of the reservoir (120)—here, a reservoir (120) in the form of a pharmaceutical cartridge (120b) with a reservoir lid (122)—thereby generating an overpressure in the vial (110); the piston's movement being guided by the piston guiding means (127/127a/127b). The piston (125) comprises a piston head (125a) where the user can place a finger to exert pressure to move the piston, and at its lower end, facing the liquid (120a) in the housing (124), a stopper (125b). FIG. 1D then shows the inserted piston (125) locked in place with the piston locking means (126), here a piston locking means (126) in the form of an engagement hook (126a) trapping the piston's head (125a), thereby storing the overpressure generated. At its lower end, the drug mixing system (10a) comprises a connection means (160), here a male connection means (160m) that allows for connection with the receiving device (200). A septum (131) is affixed to the circumferential wall (132) of the outlet port (130), here e.g., crimped-on with a metal closure, and serves as the liquid-retaining means (145) blocking liquid flow from said outlet port (130). As shown in FIG. 1E, the outlet port (130/132) is positioned in vertical alignment underneath the vial (110) and vial-piercing cannula (113) (longitudinal central axes ‘A’ and ‘P’ of the vial and the outlet port, respectively, falling in line, rather than being at an angle) to allow for unhindered, overpressure-driven, gravity-assisted, non-sinuous flow from the vial (110) towards the outlet port (130). FIGS. 1A-1E depict an embodiment in which the outlet port (130/132) protrudes, or juts out, slightly from the bottom wall of the adapter (100/100a). In alternative embodiments, the downstream end of the outlet port (130) may also be provisioned flush with the bottom wall of the drug mixing system (10/10a). A sleeve-like, dimensionally stable stabilising wall (170), equipped with dedicated sites (105) to rest four fingers of a human hand, surrounds, or encases, the adapter (100/100a) as well as the vial (110) and the reservoir (120), and thereby prevents bending of the two or dislodging them from said adapter (100/100a). Besides the piston guiding means (127), here in the form of a piston guiding groove (127a), the stabilising wall (170) also forms a piston securing means (129) at its upper end, here in the form of a small hook-like structure, which prevents loss, disconnection, or removal, of the piston (125) from the reservoir's housing (124).

FIG. 2A depicts a cross-sectional side view, or slanted rear-view, of an exemplary embodiment of a receiving device (200) according to the first aspect of the invention, more specifically a receiving device in the form of a nebulizer (201), and further specifically a vibrating mesh nebulizer (202) with a nebulizing chamber (203) and a mesh (204), or perforated disk. The receiving device (200a) depicted in FIG. 2A is adapted for combination and interaction with the seal-type drug mixing system (10a) depicted in FIG. 1A-1E by means of a dedicated lid (270a) comprising an inlet opening (210) which not only allows access into the nebulizing chamber (203) but is also equipped with a liquid-releasing means (255) in the form of a septum-piercing means (230) with a piercing tip (231). Said septum-piercing means (230) is positioned in the center of a spokes-wheel shaped part (220) that sits within the inlet opening (210). In FIG. 2A, the septum-piercing means (230) is provided as a solid piercing pin (230b). Alternatively, and as depicted e.g., in FIG. 3A-3C, the septum-piercing means (230) may also be provided as a cannula, or hollow needle (230a). An elevated wall (271) positioned on top of the lid (270/270a) and surrounding the inlet opening (210) serves as a female counter-connection means (260f) into which the lower end of the drug mixing system (10a), which serves as the corresponding male connection means (160m), can be inserted, or nestled.

FIG. 2B depicts a slanted top view of an exemplary embodiment of a lid (270/270a) of a receiving device (200a) similar to the one depicted FIG. 2A, allowing a better view onto an exemplary spokes-wheel shaped part (220) which supports the septum-piercing means (230/230b) at its center and allows for liquid to flow through the inlet opening (210), past the ribs (220a) of the spokes wheel part (220), and into the nebulizing chamber (203; not depicted here). In the depicted embodiment, the elevated wall (271) that surrounds the inlet opening (210) and serves as a female counter-connection means (260f) of the receiving device (200a) is shaped as a full circle.

FIGS. 3A-3C show how the seal-type drug mixing system (10a) depicted in FIG. 1A-1E and the receiving device (200a) in form of a vibrating mesh nebulizer (201/202) depicted in FIG. 2A interact with one another, upon inserting, or nestling, the lower end of the drug mixing system (10a) which serves as the male connection means (160m) into the corresponding female counter-connection means (260f) of the receiving device (200a/201/202) and thereby causing the piercing tip (231) of the septum-piercing means (255/230/230a) to pierce, or penetrate, the septum (131)—or in other words, actuate the liquid-releasing means (255)—and thus release the dissolved or diluted drug formulation (110b) from the vial (110), through the outlet port (130), and into the receiving device (200a/201/202) using the overpressure generated by pressing the piston (125) into the reservoir housing (124). Both the outlet port (130/132) and the receiving device's inlet opening (210) are in vertical alignment underneath the vial (110) and vial-piercing cannula, rather than at an angle to each other, to allow for unhindered, overpressure-driven, gravity-assisted, non-sinuous flow from the vial (110) through the outlet port (130/132) and the inlet opening (210) into the receiving device (200), here, into the nebulizing chamber (203), once the septum (131) is pierced.

FIGS. 4A-4D resemble FIG. 1A-1E but depict cross-sectional front views of an alternative exemplary embodiment of a drug mixing system (10) of the first aspect of the invention instead, namely, a valve-type drug mixing system (10b); i.e., a drug mixing system in which the liquid-retaining means (145) is provided in the form of a switch-valve (150), and the liquid-releasing means (155, 255) in the form of a switch-valve operating means (156).

FIG. 5 depicts a receiving device (200b) adapted for combination and interaction with the valve-type drug mixing system (10b) depicted in FIG. 4A-4D. The receiving device (200b) is essentially the same as the one depicted in FIG. 2A (e.g., a nebulizer (201), and more specifically a vibrating mesh nebulizer (202)) but is equipped with a different lid (270b) which does not comprise a septum-piercing means (230) in its inlet opening (210).

FIGS. 6A and 6B depict cross-sectional side views of the exemplary embodiment of the valve-type drug mixing system (10b) depicted in FIG. 4A-4D in interaction with the receiving device (200b) depicted in FIG. 5. The elevated wall (271) on top of the receiving device's lid (270b) that surrounds the receiving device's inlet opening (210) serves as a female connection means (260f) of the receiving device (200b), into which the lower end of the drug mixing system (10b), that serves as the corresponding male counter-connection means (160m), can be inserted, or nestled, so as to connect drug mixing system and receiving device. Moreover, the inlet opening (210) in the receiving device's lid (270b) is shaped and sized such as to receive the drug mixing system's outlet port (130/132), which protrudes, or juts out, from the bottom wall of the adapter (100/100b). Further, the valve-type drug mixing system (10b) exhibits a liquid-releasing means (155) in the form of a protrusion (156a), similar to a push-button, which juts out from the adapter's male connection means (160m) and which acts as a switch-valve operating means (156). Upon inserting, or nestling, the male connection means (160m) of the drug mixing system (10b) into the corresponding female counter-connection means (260f) of the receiving device (200b/201/202), the switch-valve operating means (156/156a) is gradually pressed inwards by the female counter-connection means (260f), thereby shifting the valve from a first valve-position which blocks liquid flow through the outlet port (130; see FIG. 6A) into a second valve-position which allows liquid flow through the outlet port (130; see FIG. 6B), and thus releasing the dissolved or diluted drug formulation (110b) from the vial (110), through the outlet port (130), and into the receiving device (200b/201/202), here, into the nebulizing chamber (203), using the overpressure generated by pressing the piston (125) into the reservoir housing (124). Both the outlet port (130/132) and the receiving device's inlet opening (210) are in vertical alignment underneath the vial (110) and vial-piercing cannula, rather than at an angle to each other, to allow for unhindered, overpressure-driven, gravity-assisted, non-sinuous flow from the vial (110) through the outlet port (130/132) and the inlet opening (210) into the receiving device once the switch-valve (150) is actuated by the switch-valve operating means (156/156a).

FIGS. 7A-7C depict side views of exemplary embodiments of the drug mixing system (10) according to the first aspect of the invention with the sleeve-like, dimensionally stable stabilising wall (170) surrounding, or encasing, most of the adapter (100), the vial (110) and the reservoir (120). Dedicated sites (105, 105′) for resting one or more finger(s) during use are provided at the stabilising wall (170); e.g., four concave indentations (105) on one side to rest four fingers of a human hand (especially during the piston moving step), as well as a patterned ledge (105′), one on either side of the drug mixing system), for resting one or two fingers (especially during the adapter activation step). FIGS. 7A and 7B show a piston protrusion (127b) that is shaped such as to ‘hug’ the corresponding piston guiding groove (127a) with flanges (127b′/127″) on both sides of the stabilising wall (170), whereas the piston protrusion (127b) in FIG. 7C is shaped with a flange (127′) only on the inner side, or against the inner surface, of the stabilising wall (170). FIG. 7B shows the inserted piston (125) locked in place with the piston locking means (126), here a piston locking means (126) in the form of an engagement hook (126a) trapping the piston's head (125a).

FIGS. 8A-8C depict side views of exemplary embodiments of the drug mixing system (10) according to the first aspect of the invention, such as the drug mixing system (10) depicted in FIG. 7C, during use. FIGS. 8A and 8B show the activation step of the adapter (100)—the adapter being covered by the outer stabilising wall (170) in the depictions—using one or two hand(s) (300), respectively, with the fingers resting on dedicated sites shaped as patterned ledges (105′), one on either side of the drug mixing system, and the thumb(s) being pressed against the flat bottom end, or lower end, of the adapter (100). Alternatively, to the finger placing shown in FIG. 8A, the user could also place the drug mixing system (10) between the splayed index-and middle finger. FIG. 8C shows the position of the hand (300) during the insertion step of the piston (125), i.e., upon pressing said piston into the housing (124) of the reservoir (120), with the thumb resting/pressing on the piston (125), or the piston's head (125a), and the four fingers resting in, or against, their respective dedicated sites shaped as four concave indentations (105) and thus holding the drug mixing system (10) firmly in the hand (300), yet without exerting any potentially displacing forces directly onto the vial (110) and/or the reservoir (120).

FIGS. 9A-9C depict side views of exemplary embodiments of the drug mixing system (10) according to the first aspect of the invention in which the piston locking means (126) is provided in the form of an engagement hook (126a), and more specifically a plurality thereof (126a, 126a′, 126a″) which trap the piston head (125a). In the depicted embodiment, there are three engagement hooks (126a, 126a′, 126a″) which are spaced such as to allow a graduated insertion and locking of the piston (125) in three steps which then transfer ⅓, ⅔ or 3/3 of the liquid (120a) housed in the reservoir (120) into the vial (110). The respective insertion depths for the piston (125) are marked on the outside of the adapter's stabilising wall (170) by visible markings (173), here shown in the form of raised markings. Unlike in FIGS. 1, 3, 4, and 6-8, where the engagement hook (126a) was shaped at/in a piston guiding groove (127a), the embodiments depicted in FIG. 9A-9D show engagement hooks (126a, 126a′, 126a″) which jut out from a piston guide rail (127aa). The piston (125), and more specifically the piston head (125a) is equipped with a corresponding piston guiding notch (127bb) which is shaped such as to ‘hug’ the piston guide rail (127aa) while the user presses the piston (125) down into the housing (124). Further, in the embodiments depicted in FIG. 9A-9C, the piston (125) is not locked in position by trapping the piston head's upper surface (where the user would rest a finger) under one of the engagement hooks (126a, 126a′, 126a″); instead a resilient, or springy, tailhook (126b) that is fixedly associated with, or forming an integral part of, the piston head (125a) is trapped under the engagement hooks. This detail is shown in more detail in the cross-sectional side view of FIG. 9D. Once the piston (125) is inserted deep enough for the tailhook (126b) to spring into one of the engagement hooks (126a, 126a′, 126a″), the user will get a tactile and/or an acoustic feedback; a small ‘click’ sensation and/or sound, and the down-ward slanted shape of the engagement hooks (126a, 126a′, 126a″) ensures that the piston (125) can only be inserted further down but not slide back unintentionally; thus, storing the overpressure generated in the vial (110) by the transfer of the liquid (120a) housed in the reservoir (120); here, a reservoir (120) in the form of a pharmaceutical cartridge (120b). As is also visible from FIG. 9D, there are two sets of both the piston guiding means (127/127a/127b) and the piston locking means (126, 126a, 126a′, 126a″, 126b), one on either side of the adapter (100) and the piston head (125a). As is further visible from FIG. 9D, the elevated wall (271) that is positioned on top of the receiving device's lid (270) (receiving device not shown here) serves as a male counter-connection means (260m) which is inserted into the corresponding female connection means (160f) on the lower end of the drug mixing system (10a). As can be seen in FIG. 9B, the outlet port (130) and its circumferential wall (132) are positioned in vertical alignment under-neath the vial (110), rather than at an angle to each other, to allow for unhindered, overpressure-driven, gravity-assisted, non-sinuous flow from the vial (110) towards the outlet port (130). The embodiments depicted in FIG. 9A-9C further show a small indicator window (172) which indicates to the user, typically in a visual manner, whether the adapter (100) is in its activated position (i.e., whether the two adapter subunits (not shown in detail in FIG. 9) have been moved towards each other and thus the vial (110) and reservoir (120) pierced). The indicator window (172) could turn green, for instance, once the adapter (100) is activated. Alternative embodiments to those depicted in FIG. 9A-9D could comprise, for instance, different graduations (e.g., ½ and 2/2 instead of thirds), printed or engraved markings instead of raised ones, and/or a tactile activation indicator instead of a colour-changing one.

FIGS. 10A-10D depict further exemplary embodiments of the drug mixing system (10) according to the first aspect of the invention, in particular the use in connection with a receiving device (200) in the form of a nebulizer (201), more specifically a vibrating mesh nebulizer (202). Typically, the user is provided with both the nebulizer (201/202) and the drug mixing system (10) sterile-packaged in preassembled form in package (400), as shown in FIG. 10A; optionally in the form of a starter kit. Later, the nebulizer (201/202) is typically re-used with fresh, separately packed drug mixing systems (10) being provided for preparing the drug formulation (110b) afresh prior to each use. As shown in FIG. 10A, the user removes the protective packaging (400) housing the drug mixing system (10) in a sterile environment until use, including its adapter (100), the vial (110) storing the drug formulation (110a), and the reservoir (120) housing the liquid (120a) for dissolution or dilution thereof; with the vial (110) and reservoir (120) already inserted and held firmly in the adapter to prevent, or reduce the risk of, contamination and/or faulty assembly. In a first handling step, shown at the top of FIG. 10B, the user activates the adapter (100), i.e., moving the two adapter subunits (not shown in detail in FIG. 10) towards each other, by pressing against the lower end of the drug mixing system (10) and the bottom (115) of the vial (110), with activation being noticeable via a tactile and/or an acoustic feedback (a small ‘click’ sensation and/or sound) as well as by a colour change in the indicator window (172). Thereafter, the piston (125) is inserted into the reservoir housing (124) by manually pressing onto the piston head (125a), moving the piston to either just the first through-hole (126c) or to the second through-hole (126c′) in order to transfer either one half or both halves of the liquid (120a) from the reservoir (120) into the vial (110), respectively (as detailed further below) and generating a corresponding overpressure. Using the viewing window (171), the user can then visually assess when the drug formulation (110a) stored in the vial (110) is dissolved or diluted to the desired extent, as shown in center of FIG. 10C (here indicating the formation of a solution); optionally aiding the process by lightly shaking or swirling the drug mixing system (10). Thereafter, the lower end of the drug mixing system (10) is placed onto the lid (270) of the nebulizer (201/202), and lightly pressed down (e.g., pressing onto the bottom (115) of the vial (110) again) to actuate the liquid-releasing means and thereby expel, or transfer, the dissolved or diluted drug formulation (110b) from the vial (110) through the outlet port (130) using the generated overpressure. The actuation of the liquid-releasing means is noticeable via a tactile and/or an acoustic feedback (a small ‘click’ sensation and/or sound) as well as visually through the viewing window (171) of the vial (110). After the dissolved or diluted drug formulation (110b) is transferred from the vial (110) into the nebulizer (201/202), the drug mixing system can be removed, and the nebulizer is ready for inhalation as indicated in FIG. 10D. As further shown in FIG. 1D, center, in the depicted embodiment, the outlet port (130), or more specifically the downstream end thereof, is provisioned flush with the bottom wall of the drug mixing system (10).

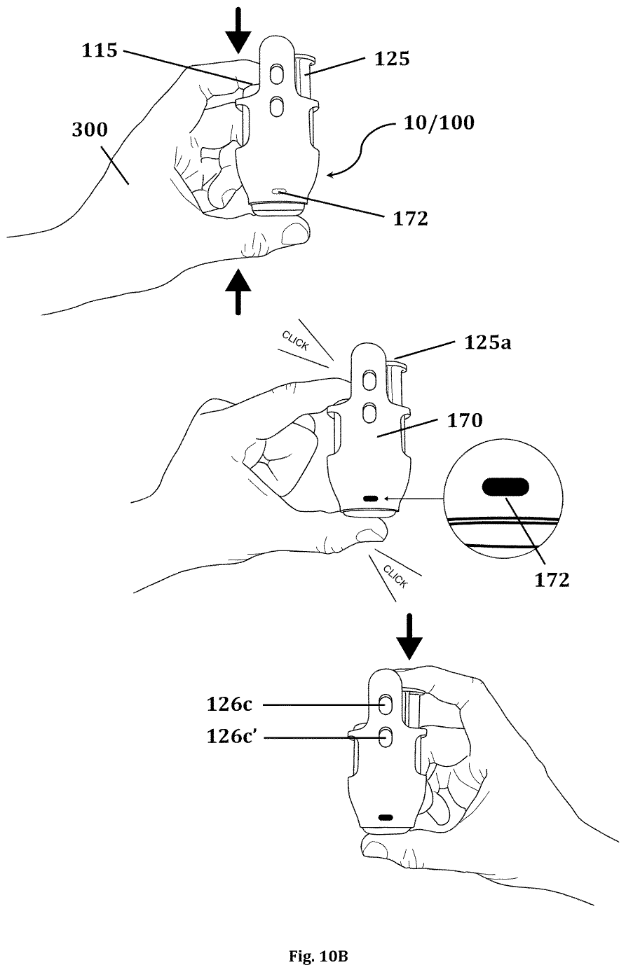

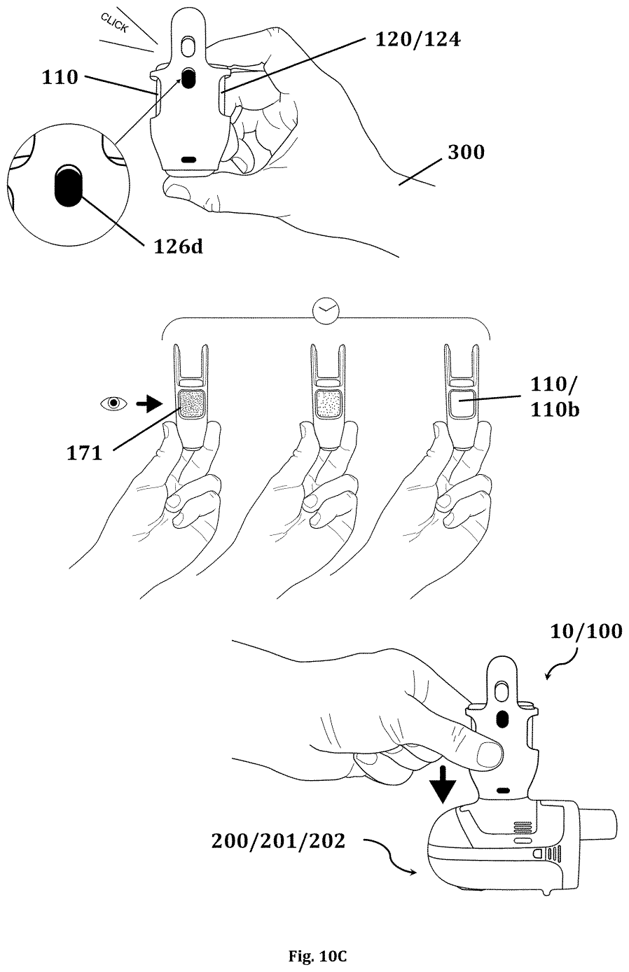

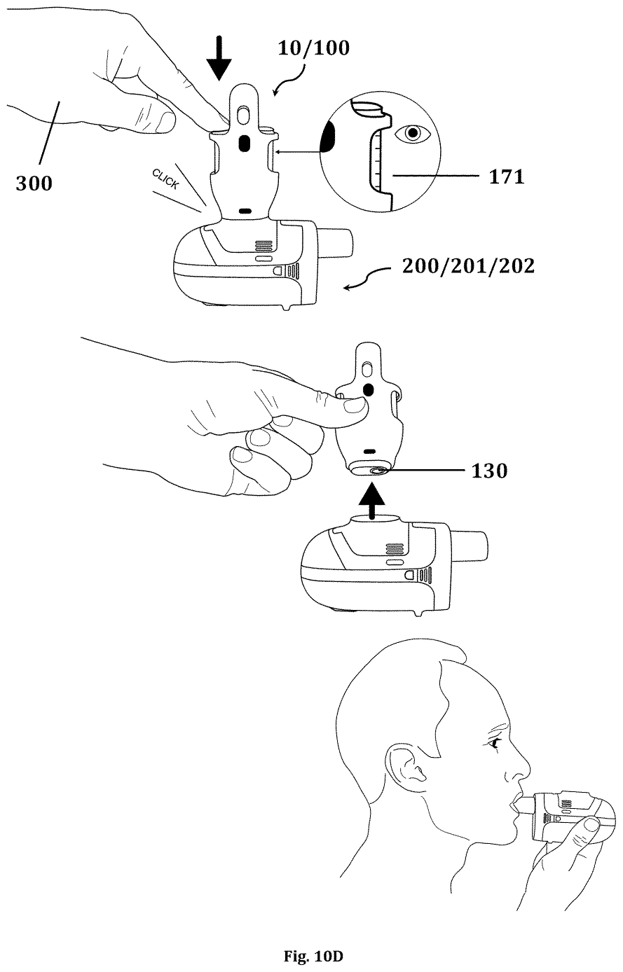

As yet further shown in FIG. 10A-10D, the depicted embodiment comprises an alternative piston locking means (126), namely a piston locking means provided in the form of a snap-fit locking mechanism (126c), or a plurality thereof. Here, the adapter's stabilising wall (170) exhibits two through-holes (126c, 126c′) into which a corresponding piston locking protrusion (126d) of appropriately adapted size and shape, positioned at the piston head (125a), can snap-fit when reaching the position of the through-hole(s) upon insertion of the piston (125); see e.g., top of FIG. 10C. The snap-fit is noticeable via a tactile and/or an acoustic feedback; a small ‘click’ sensation and/or sound. In addition, the piston locking protrusion (126d) at the piston head (125a) may also be of a different colour than the material of the stabilising wall (170) so as to show visually once snap-fitted into one of the two through-holes (126c, 126c′), as shown in FIG. 10C. The provision of two through-holes is meant to allow for a graduated piston insertion and locking in half-steps, i.e., allowing the transfer of one half or both halves of the liquid (120a) housed in the reservoir (120) into the vial (110).

### DEFINITIONS

The following expressions as used herein should normally be interpreted as outlined in this section, unless the description provides a different meaning in a specific context.

The terms “a”, “an” or “the” do not exclude a plurality; i.e., these singular forms should be understood such as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms “a”, “an” or “the” thus have the same meaning as “at least one” or as “one or more” unless defined otherwise.

The expressions “one embodiment”, “an embodiment”, “a specific embodiment” and the like mean that a particular feature, property or characteristic, or a particular group, or combination, of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

Terms such as “about”, “approximately”, “ca.”, “essentially”, or “substantially” are meant to compensate for the variability allowed for in the technical field concerned and inherent in the respective products (e.g., in the pharmaceutical industry and in pharmaceutical products), such as differences in content due to manufacturing variation and/or time-induced product degradation. The terms in connection with an attribute or value include the exact attribute or precise value, as well as any attribute or value typically considered to fall within a normal range or variability accepted in the technical field concerned.

The term “comprise” is to be construed in an open and inclusive sense, as “including, but not limited to”. The expressions “substantially consisting of” or “essentially consisting of” mean that no further components are added other than those listed.

The term “unreleasable” is understood to mean that the respectively connected components of the adapter or the drug mixing system are not releasable, or separable, from one another without requiring brute force and/or without breaking the adapter or the drug mixing system.

The term “injectable liquid” is understood as any fluid that is able to flow and that is suitable and safe for parenteral administration to a patient or animal.

Terms such as “drug”, “active agent”, “active pharmaceutical ingredient” (API), or the like are used synonymously herein and refer to a compound or combination of compounds which are pharmaceutically active against an undesired condition.

The term “pharmaceutically acceptable” means that a material is useful in preparing a pharmaceutical composition that is generally safe, non-toxic and exhibits neither biologically nor otherwise undesirable properties which would prevent, or exclude, it from pharmaceutical use in humans.

Terms designating a position, orientation, or direction, such as left, right, front, rear, back, top, bottom, up, down and the like, should be understood with reference to the orientation of the disclosed devices or their components under normal operational conditions (i.e., use as intended), and typically from the perspective of the user.

A single unit may fulfil the functions of several features recited in the claims.

### DETAILED DESCRIPTION OF THE INVENTION

In a first aspect, the invention relates to a drug mixing system (10) for dissolving or diluting a drug formulation (110a) stored in a vial (110) prior to use, the drug mixing system (10) comprising an adapter (100) adapted to hold the vial (110) housing the drug formulation (110a), and a reservoir (120) for housing a liquid (120a) for dissolution or dilution of the drug formulation (110a), the reservoir (120) comprising a housing (124) and a piston (125) sealingly movable within the housing, wherein the adapter (100) comprises a vial mounting opening (111) adapted to hold the vial (110), a reservoir mounting opening (121) adapted to hold the reservoir (120), an outlet port (130), an internal adapter channel (140) fluidly connecting the vial-and reservoir mounting openings (111, 121), and the outlet port (130), and a liquid-retaining means (145) adapted to block at least a flow of dissolved or diluted drug formulation (110b) through the outlet port (130), characterized in that the adapter (100) is equipped with a piston locking means (126) adapted to lock the position of the piston (125) once a predetermined volume of the liquid has been transferred from the reservoir (120) into the vial (110) by means of pressing the piston (125) into the housing (124), thereby creating and storing an overpressure, and in that the drug mixing system (10) is adapted to expel, or transfer, the dissolved or diluted drug formulation (110b) from the vial (110) through the outlet port (130) upon actuation of a liquid-releasing means (155, 255) using the overpressure generated by pressing the piston (125) into the housing (124).

The drug mixing system (10) according to this first aspect may be provided in at least two different designs which will be referred to herein as either (a) a seal-type drug mixing system (10a); or (b) a valve-type drug mixing system (10b), with the difference between these two designs mainly residing in their liquid-retaining means (145) and their liquid-releasing means (155, 255); i.e., (i) how the flow of a liquid through the outlet port (130), for instance, the flow of the dissolved or diluted drug formulation (110b), is prevented, or held back, while the overpressure is being generated; and (ii) how said flow is then enabled once the liquid is ready to be expelled, or transferred, through the outlet port (130).

For the seal-type drug mixing system (10a), the liquid-retaining means (145) is provided in the form of a septum (131) covering the outlet port (130), and the liquid-releasing means (155, 255) is provided in the form of a septum-piercing means (230) so that, upon actuation, the septum (131) is pierced by the septum-piercing means (230) and the dissolved or diluted drug formulation (110b) is expelled, or transferred, from the vial (110) through the outlet port (130) using the over-pressure generated by pressing the piston (125) into the housing (124). The septum (131) can be provided in a variety of sealing materials as commonly used, for instance, to close vials containing lyophilized drugs, (e.g., rubber, silicone, blends of the two with polytetrafluoroethylene (PTFE), chlorbutyl, bromobutyl, or the like), and can be affixed to the circumferential wall (132) forming the outlet port (130) in any manner that precludes its separation from said outlet port (130) under pressure (e.g., crimped-on with a metal closure, or affixed tightly by heat-staking).

For the valve-type drug mixing system (10b), the liquid-retaining means (145) is provided in the form of a switch valve (150), and the internal adapter channel (140) and the switch valve (150) are adapted to at least allow selective fluid connection between the vial (110) and the reservoir (120) while blocking fluid connection to the outlet port (130) when in valve position 1, and between the vial (110) and the outlet port (130) while blocking fluid connection to the reservoir (120) when in valve position 2; and the liquid-releasing means (155, 255) is provided in the form of a switch-valve operating means (156) adapted to, upon actuation, bring the switch valve (150) into valve position 2, so that, upon the switch valve (150) being brought into said valve position 2, the dissolved or diluted drug formulation (110b) is expelled, or transferred, from the vial (110) through the outlet port (130) using the over-pressure generated by pressing the piston (125) into the housing (124). In one embodiment, the switch valve (150) is a three-way switch valve having three openings to selectively enable fluid connection between the vial-mounting opening (111), the reservoir-mounting opening (121), and the outlet port (130). In a further embodiment, the switch valve (150) is rotatable or shiftable, i.e., it may be operated using a rotatable button, or a push-button (156), to move the switch valve (150) from position 1 to position 2. Optionally, in a specific embodiment, the switch valve (150) can be adapted to also switch to a third position, valve position 3, blocking any fluid connection between the vial-mounting opening (111), the reservoir-mounting opening (121), and the outlet port (130).

As depicted in the figures, the piston (125) comprises, (i) at its upper end and outside the reservoir housing (124), a piston head (125a), i.e., the part where a user of the drug mixing system (10) can place a finger to exert pressure to move the piston into the housing, and (ii) at its lower end, facing the liquid (120a) stored in the housing (124), a stopper (125b) which ensures a sealing movement of the piston (125) within the housing, (124) so that the liquid (120a) can be pushed from the reservoir (120) into the vial (110), generating an overpressure. According to the invention, the piston (125) is pressed towards and into the housing, not vice versa. This is beneficial insofar as the piston (125), and in particular its piston head (125a), can be more easily modified than the housing (124); the latter often requiring specific, fixed dimensions to ensure the defined volumes to be contained therein. In one of the preferred embodiments, the piston head (125a) is adapted such as to allow physical interaction with both the adapter's piston locking means (126) as well as a piston guiding means (127), as will be explained in more detail below.

In one of the further preferred embodiments, the piston head (125a) is adapted such as to be attachable to the piston (125), e.g., attachable as a snap-on or slip-on ‘crown cap’, thereby allowing, for instance, standardized pistons such as those used in commercially available syringes or pharmaceutical cartridges to be customized for use with the drug mixing system (10) according to the first aspect of the invention.

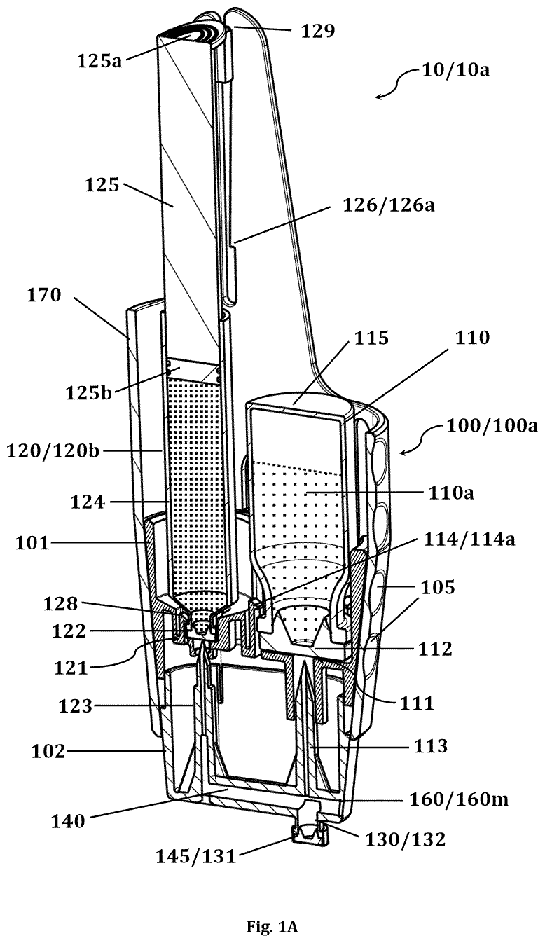

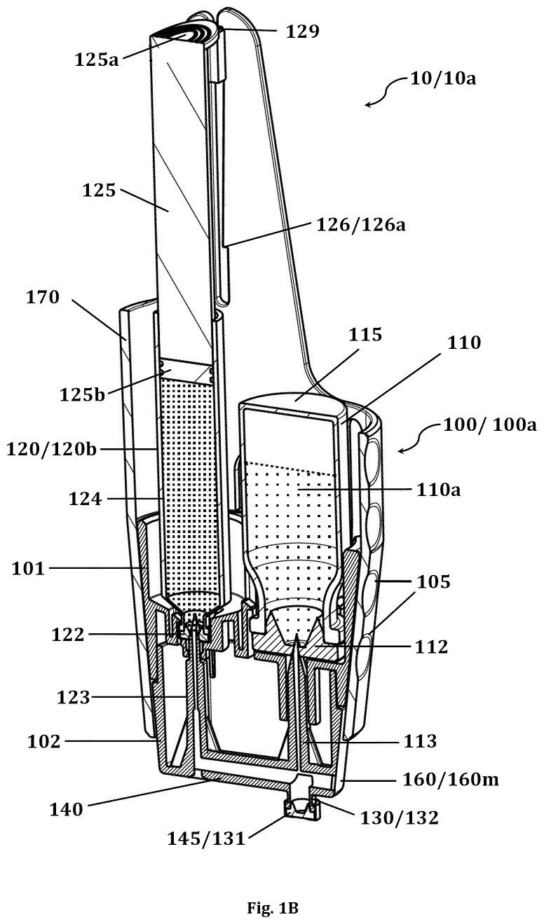

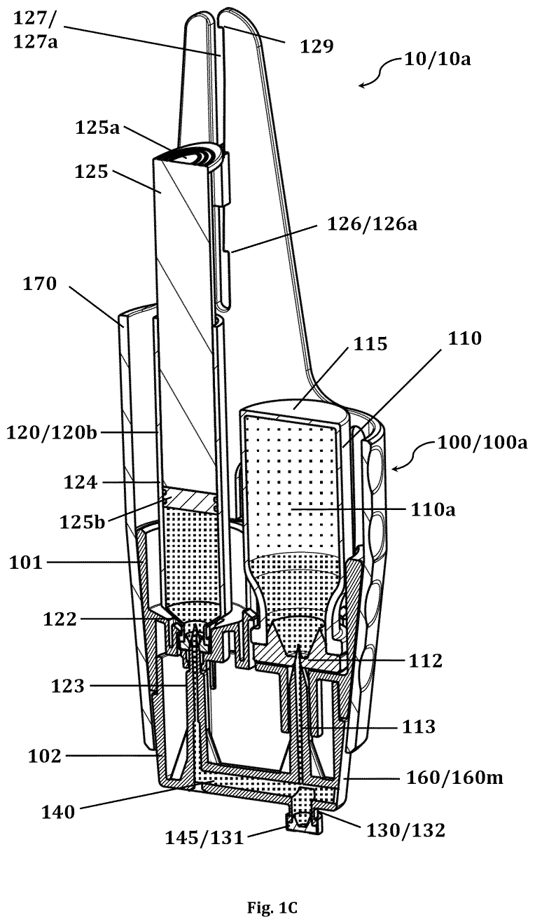

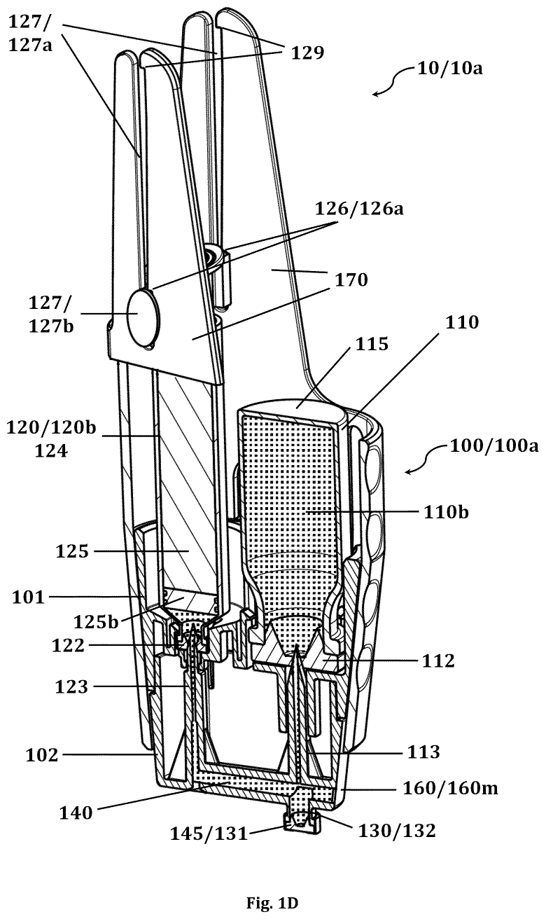

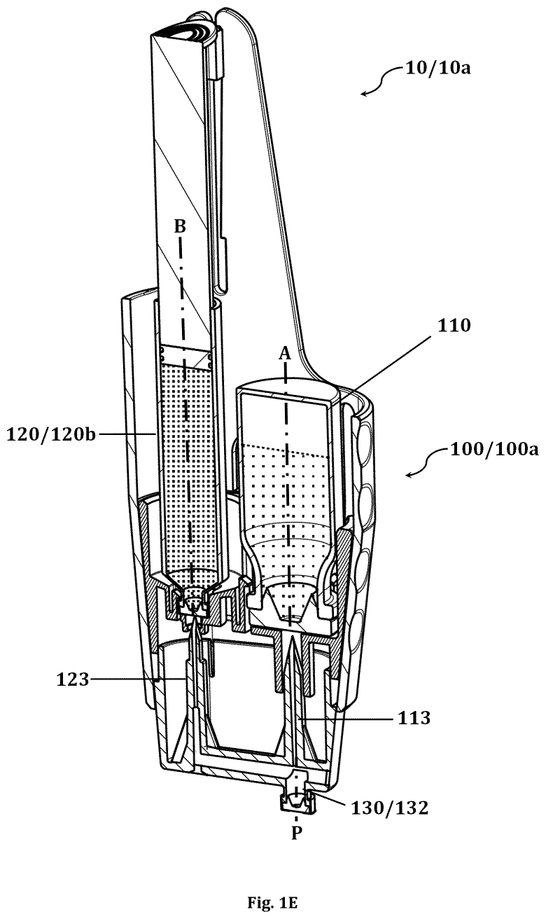

Moreover, according to the invention, the outlet port (130) and its circumferential wall (132) are positioned in vertical alignment with the vial (110) and underneath the vial (110) (rather than being positioned at an angle to each other) to allow for unhindered, overpressure-driven, gravity-assisted, non-sinuous flow from the vial (110) towards the outlet port (130). Vertical alignment, as used herein, refers to the case where the vial (110) exhibits a longitudinal central axis ‘A’ and the outlet port (130) exhibit a longitudinal central axis ‘P’ and the two axes fall in line with one another (as exemplarily depicted in FIG. 1E), or axes ‘A’ and ‘P’ are at least parallel to each other and only a short distance of no more than 5 mm, preferably no more than 2 mm, off-set from one another. Any flow-guiding structures placed between the vial (110) and the outlet port (130), such as the vial-piercing cannula (113) depicted in FIG. 1E, shall preferably also be positioned in vertical alignment with and underneath the vial (110), and be straight, non-collapsible, and non-tortuous so as to not hinder the overpressure-driven, gravity-assisted, non-sinuous flow from the vial (110) towards the outlet port (130).

Further details on both systems, the seal-type drug mixing system (10a), and the valve-type drug mixing system (10b), will be provided below.

The drug mixing system (10) according to the first aspect of the invention can be employed in a wide variety of purposes requiring the preparation of fresh liquid drug formulations right before use; examples thereof including, but not being limited to, drugs that are sensitive to hydrolysis and thus need to be stored in dry state but administered in liquid form. The dissolved or diluted drug formulation (110b) formed in the vial (110) can, for instance, be applied directly from the drug mixing system (10) to the skin of a user. More commonly, though, for the purpose of administering the dissolved or diluted drug formulation (110b) formed inside the vial (110) is transferred from said vial (110) and via the outlet port (130) into a receiving device (200) for receiving the dissolved or diluted drug formulation (110b). The receiving device (200) can be any suitable administration device for liquid drug formulations, for instance, infusion devices such as syringe drivers (also known as perfusors, or perfusion pumps) or nebulizers.

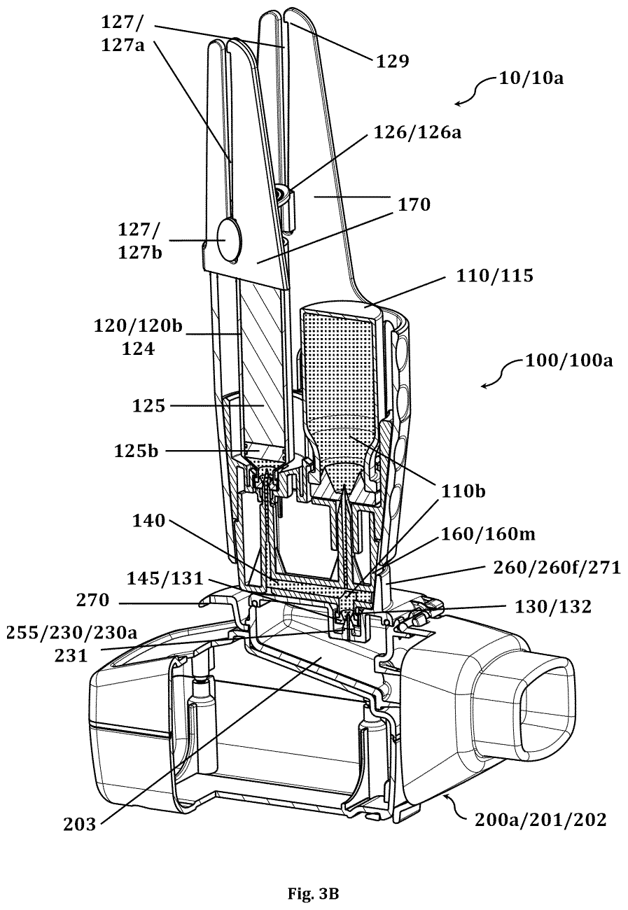

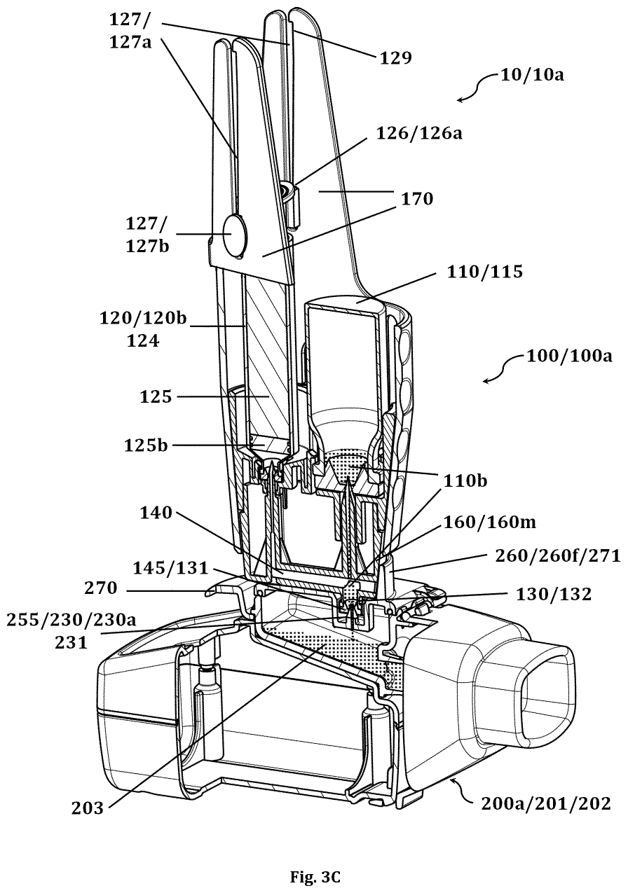

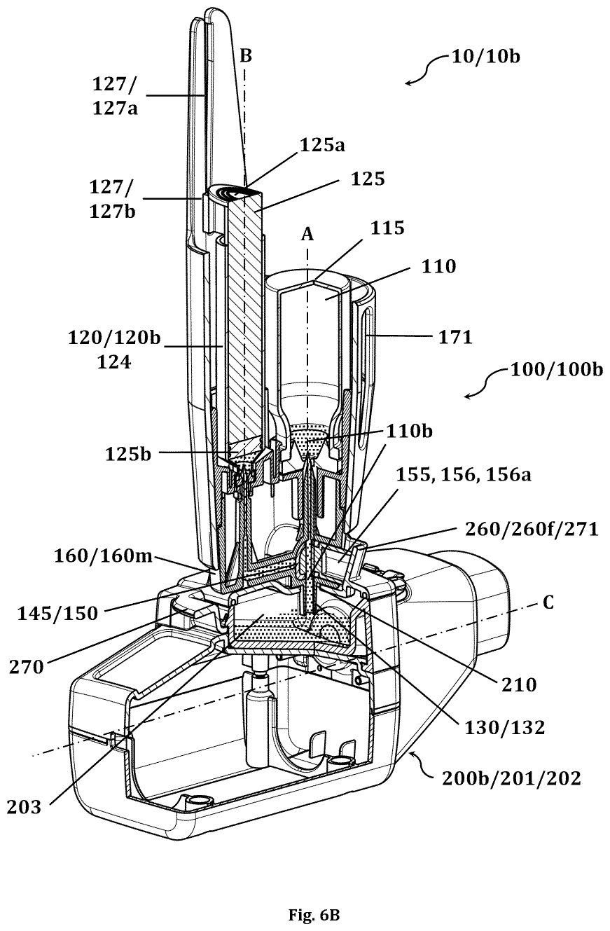

In one embodiment, the receiving device (200) is a nebulizer (201). In a more specific embodiment, the receiving device (200) is a vibrating mesh nebulizer (202) with a nebulizing chamber (203; or drug reservoir) and a mesh (204; or perforated plate). Here, the dissolved or diluted drug formulation (110b) from the vial (110) is transferred into, or received by, the nebulizing chamber (203) of the vibrating mesh nebulizer (202), as depicted in e.g., FIG. 3C or 6B.

The liquid-releasing means (155, 255) can be provided as a part, optionally as an integral part, of the receiving device (200) or of the adapter (100). This means that, in embodiments including a receiving device (200), the dissolved or diluted drug formulation (110b) is then released from the vial (110) upon an interaction of the drug mixing system (10) with the receiving device (200).

For the purpose of connecting the drug mixing system (10) with the receiving device (200), the adapter (100) and the receiving device (200) are respectively equipped with connection means (160) and counter-connection means (260), with reference sign ‘160’ referring to connection means provided at the adapter (100), and ‘260’ referring to connection means provided at the receiving device (200). Such connection/counter connection means (160, 260) may, for instance, include plug-in connections, snap-fit mechanisms, or the like. In one embodiment, the connection means (160) and counter-connection means (260) are adapted in such a way that, upon connecting them, the drug mixing system (10) and the receiving device (200) form a leak-proof connection. This is preferred so as to prevent both spillage of the dissolved or diluted drug formulation (110b) and contamination of surfaces and/or the hands (300) of the user of the drug mixing system (10) and the receiving device (200) upon transfer of the dissolved or diluted drug formulation (110b).

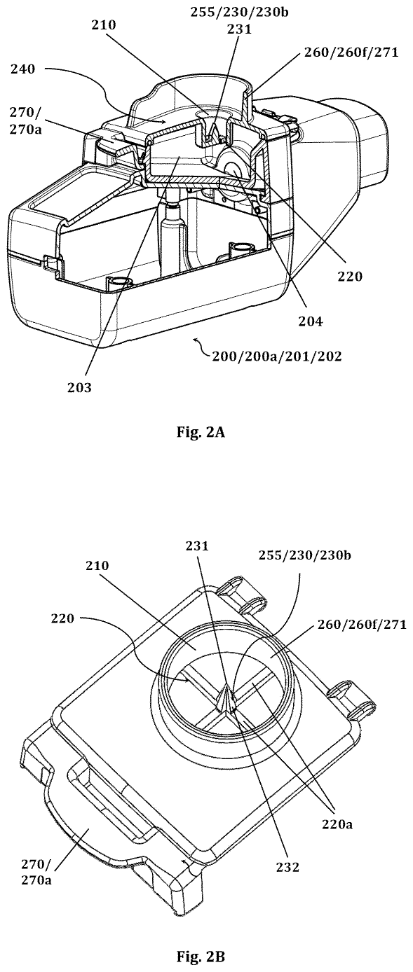

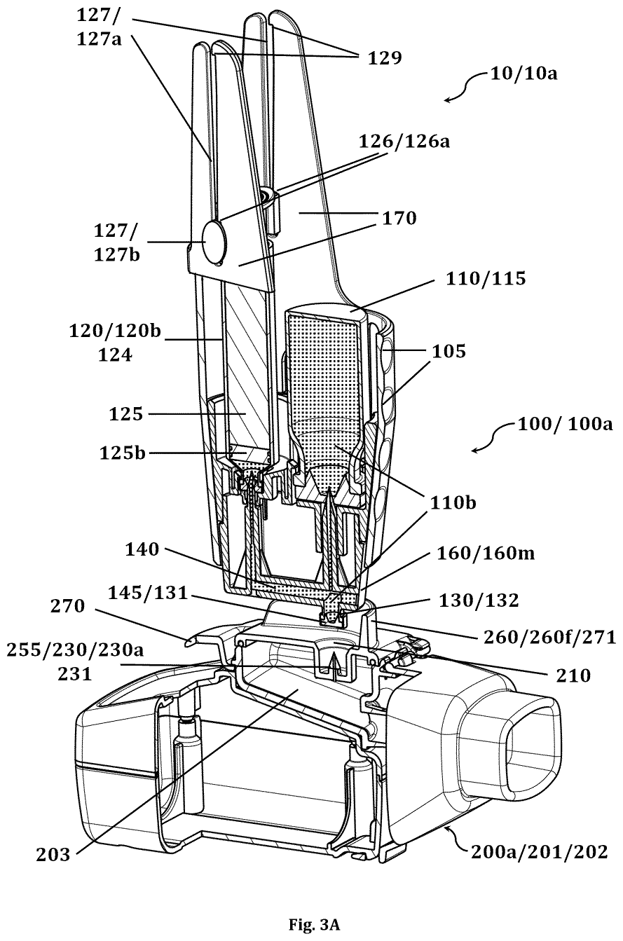

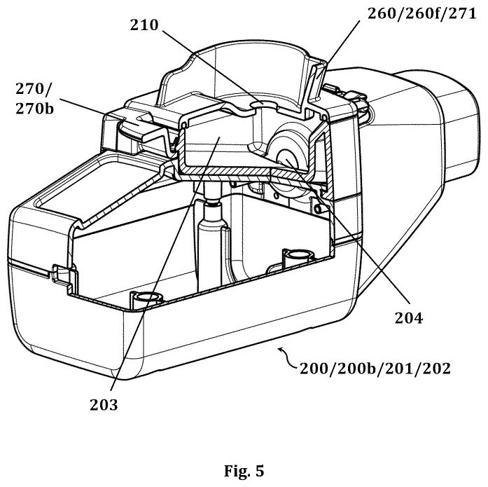

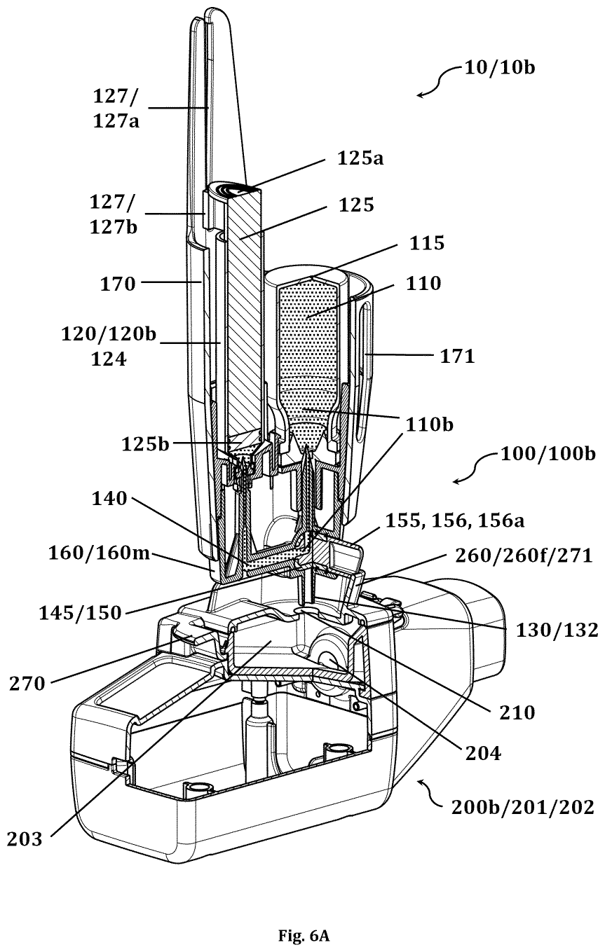

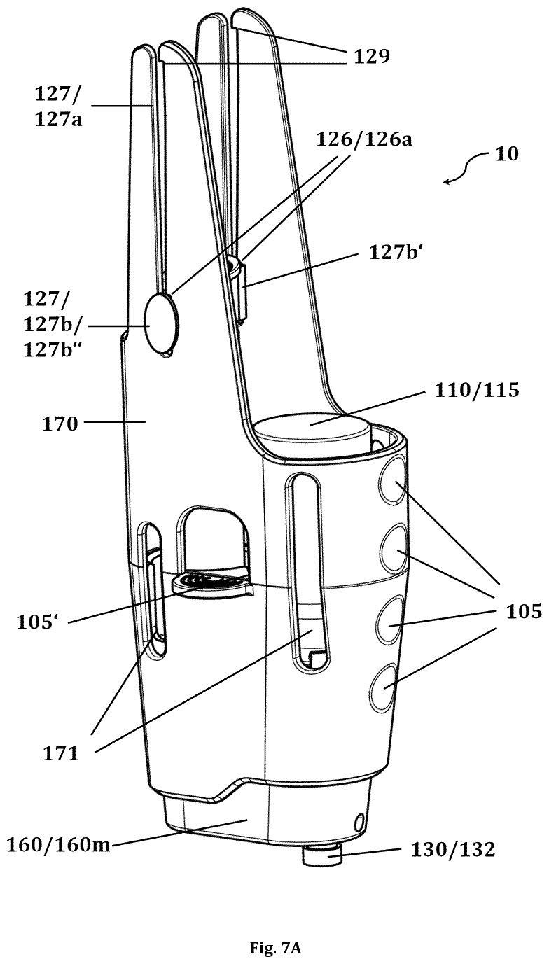

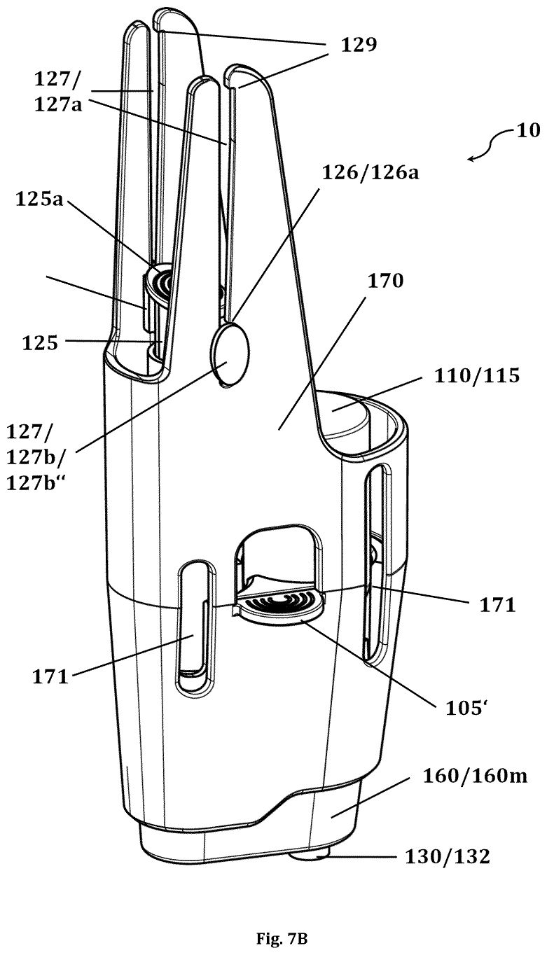

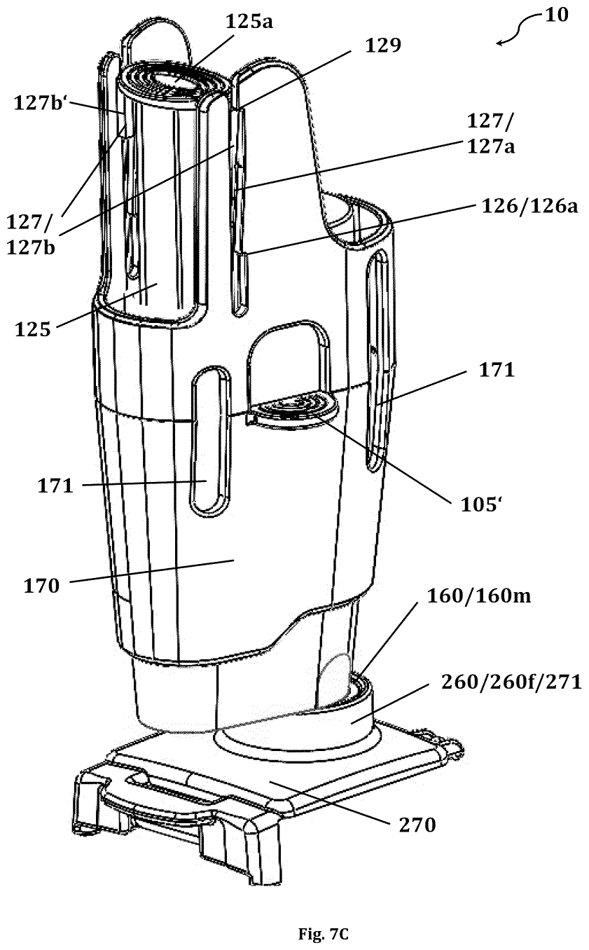

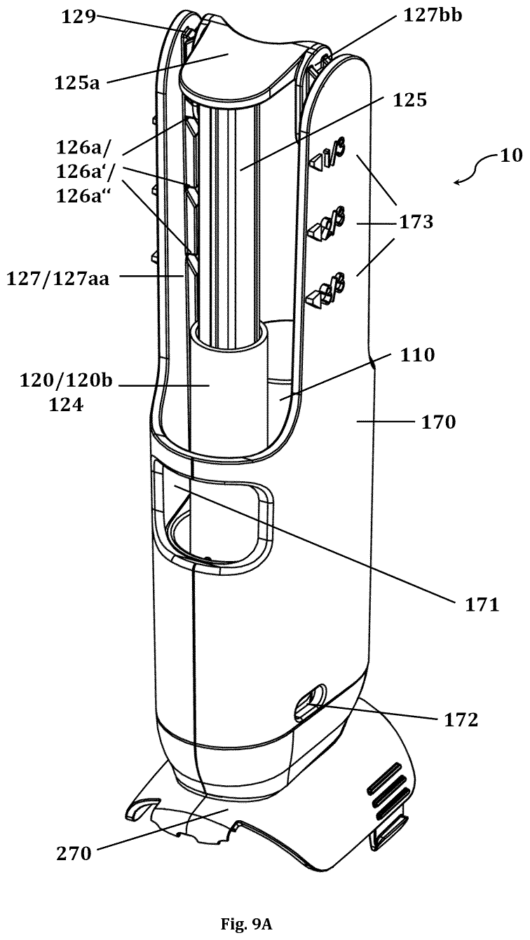

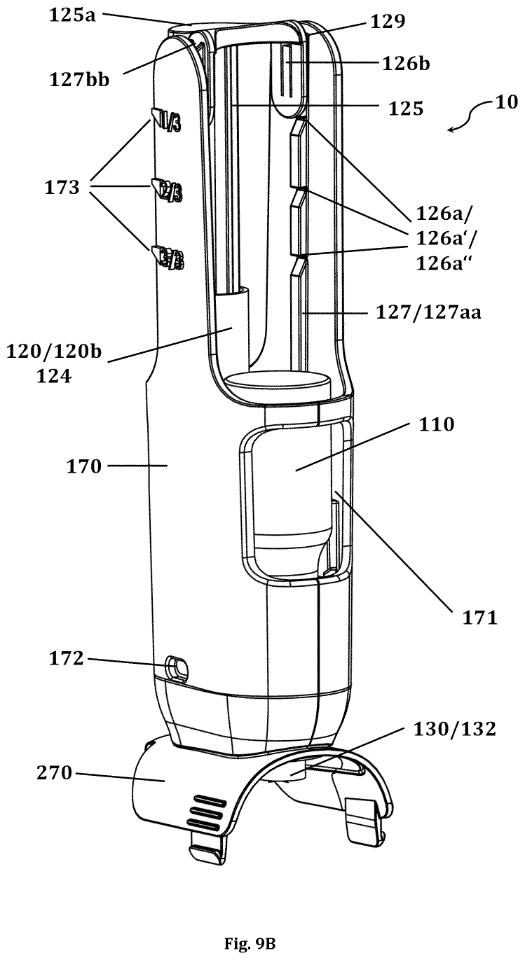

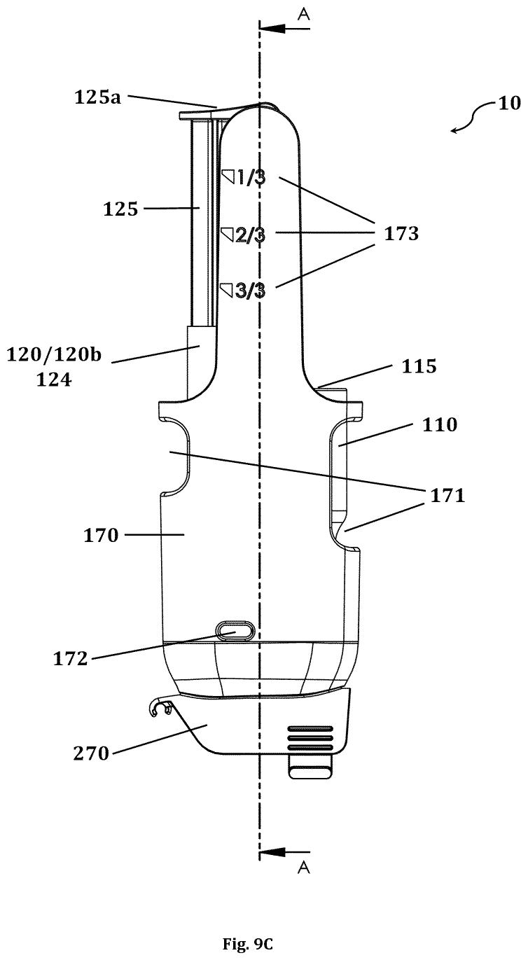

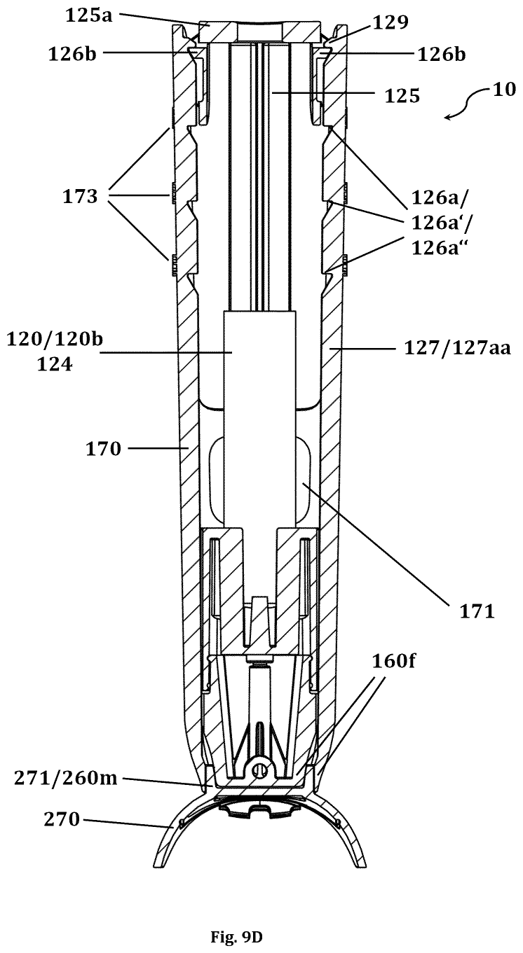

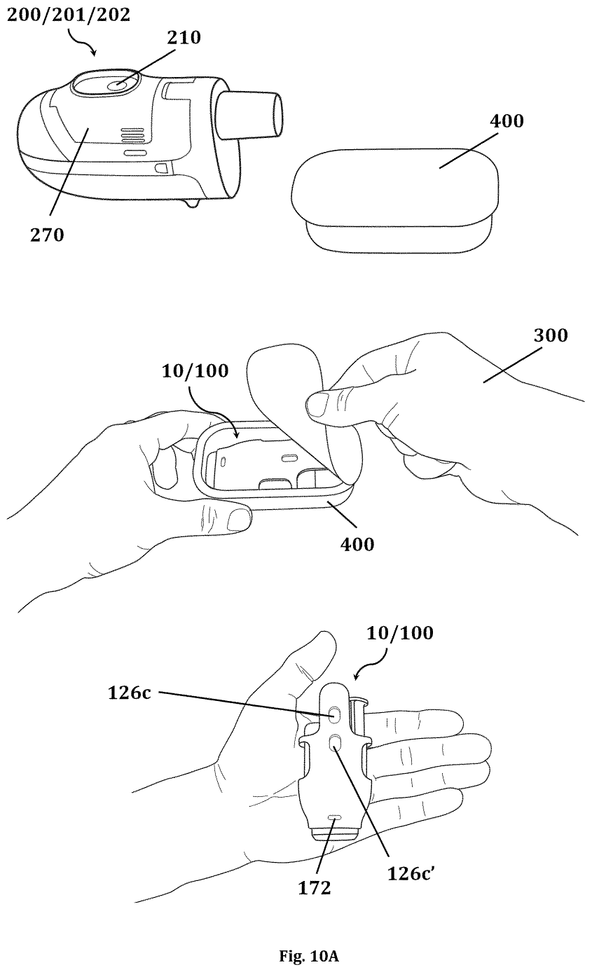

In one embodiment, the adapter (100) is equipped with a male connection means (160m) that is insertable into a female counter-connection means (260f) of the receiving device (200). For instance, as depicted in e.g., FIG. 3A-3C or 6A-6B, in one embodiment, an elevated wall (271) positioned on top of the lid (270) and surrounding the inlet opening (210) of the receiving device serves as a female counter-connection means (260f) into which a lower end of the drug mixing system (10), more specifically the lower end of the adapter (100), which serves as a corresponding male connection means (160m), can be inserted, or nestled. The precise shape of the elevated wall (271) on the lid (270) in said embodiment is of little importance as long as it matches the size and shape of the corresponding lower end of the adapter and allows for said end to be inserted, or nestled, into the shape of the elevated wall (271) with a snug fit. The elevated wall (271) may, for instance, be shaped as a complete circular wall (as e.g., in FIG. 2B or FIG. 7C) or be open on one side (i.e., approx. U-shaped). Since the lid (270) on the receiving device (200) can be provided in a detachable form, e.g., a hinged lid as shown in e.g., FIG. 2A-B, FIG. 3A-C, FIG. 5, FIG. 6A-B, or FIG. 9C, both the lid (270) and the elevated wall (271) provided thereon can be easily customized to the respective drug mixing system (10) the receiving device (200) is meant to be used with.

In that regard, it should be understood that, in some embodiments, the connection/counter connection means (160, 260) may be features that provide functionalities in addition to serving as connection means. For instance, as depicted e.g., in FIG. 6B, the circumferential wall (132) forming the outlet port (130) may jut out from the adapter (100/100b) and be shaped and sized in such a way as to fit like a ‘plug’ into a respectively shaped and sized inlet opening (210) of the receiving device (200). The circumferential wall (132) and the inlet opening (210) thus provide male/female connection means (160m, 260f) in addition to the male/female connection means (160m, 260f) provided respectively in the form of the lower end of the adapter (100/100b) and the elevated wall (271) on top of the receiving device's lid (270) surrounding the inlet opening (210). At the same time, said circumferential wall (132) may also be the part of the adapter (100/100b) to which—in seal-type drug mixing systems (10a)—the septum (131) is affixed (e.g., by a crimped on metal closure, or affixed by heat-staking) so as to block the outlet port (130).

It should further be understood that, for the purpose of connecting the drug mixing system (10) with the receiving device (200), the adapter (100) and the receiving device (200) may also be equipped with more than one connection means (160) and counter-connection means (260). For instance, the adapter (100) and the receiving device (200) may be equipped with the above-mentioned ‘plug-in’ connection between (i) the circumferential wall (132) forming the outlet port (130) and the receiving device's (200) inlet opening (210) and (ii) between the lower end of the adapter (100) and the elevated wall (271) on the lid (270) surrounding inlet opening (210), and in addition thereto may be equipped with a further snap-fit connection between the adapter (100) and the receiving device (200). In a specific embodiment, either one or both these connections may be unreleasable (e.g., a very tight snap-fit that can only be released by destruction of the connection means). This can be advantageous, for instance, for single-use drug mixing systems (10) to prevent user attempts of re-use.

In one of the preferred embodiments, the connection means (160) and counter-connection means (260) are adapted in such a way that, upon connecting them—or, in other words, upon connecting the adapter (100) of the drug mixing system (10) with the receiving device (200)—the liquid-releasing means (155, 255) is actuated. This means that, upon connection, either the septum (131) of a seal-type drug mixing system (10a) is pierced open, or the switch-valve (150) of a valve-type drug mixing system (10b) being switched from its position 1 (blocked outlet port flow) to its position 2 (enabled outlet port flow). In one of the further preferred embodiments, this actuation happens automatically, i.e., the connection means (160) and counter-connection means (260) are adapted in such a way that upon connecting the seal-type drug mixing system (10a) and the receiving device (200), the septum-piercing means (230) automatically pierces the septum (131); or that upon connecting the valve-type drug mixing system (10b) and the receiving device (200), the switch valve (150) is automatically brought into valve position 2 (i.e., without the handling person, or user, having to actively do any further steps); preferably by a physical interaction of device components. For instance, in one embodiment, upon connecting the valve-type drug mixing system (10b) and the receiving device (200), the switch valve (150) is automatically brought into valve position 2 via physical interaction of the connection means (160) and/or the counter-connection means (260) with the switch-valve operating means (156) of the adapter (100b).

In a specific embodiment, said switch-valve operating means (156) is a part of the adapter (100b) of the valve-type drug mixing system (10b). In a further specific embodiment, as depicted in e.g., FIGS. 6A and 6B, the switch-valve operating means (156) is provided in the form of a protrusion (156a), similar to a push-button, jutting out from a male connection means (160m) of the adapter (100b), wherein said male connection means (160m) is insertable into a female counter-connection means (260f) of the receiving device (200), and upon inserting the male connection means (160m) into the female counter-connection means (260f) the protruding switch-valve operating means (156a) is pressed into the male connection means (160m), thereby bringing the switch valve (150) into valve position 2 and expelling, or transferring, the dissolved or diluted drug formulation (110b) from the vial (110) via the outlet port (130) into the receiving device (200) using the overpressure.

In one embodiment, as depicted in e.g., FIGS. 6A and 6B, the adapter's (100b) protruding switch-valve operating means (156a) and/or the female counter-connection means (260f) of the receiving device (200) are also provided with angled, or sloped, surfaces to facilitate a controlled, steady movement of the protruding switch-valve operating means (156a) when being pressed into the male connection means (160m). In a specific embodiment, the protruding switch-valve operating means (156a) the adapter (100b) is covered by a protective wall, or protective cover (not depicted) that is adapted and positioned such as to prevent premature and/or unintentional movements of the protruding switch-valve operating means (156a) prior to connecting the drug mixing system (10) with the receiving device (200). This is beneficial insofar as users would not unintentionally move the valve (150) with their fingers even if they grabbed, or held, the drug mixing system (10) by the outlet port (130) end thereof. In a further embodiment, the receiving device (200) is equipped with a valve-operating pin (256; not shown), and upon inserting the male connection means (160m) of the adapter (100b) into the female counter-connection means (260f) of the receiving device (200), the protruding switch-valve operating means (156a) is pressed into the male connection means (160m) by said valve-operating pin (256), thereby bringing the switch valve (150) into valve position 2 and expelling, or transferring, the dissolved or diluted drug formulation (110b) from the vial (110) via the outlet port (130) into the receiving device (200) using the overpressure. As mentioned above, the liquid-releasing means (155, 255) can be provided as a part, optionally as an integral part, of the receiving device (200) or of the adapter (100). In one embodiment of a seal-type drug mixing system (10a)—i.e., where the liquid-releasing means (155, 255) is provided in the form of a septum-piercing means (230)—said septum-piercing means (230) is provided as a part, optionally as an integral part, of the receiving device (200). In a specific embodiment, as depicted e.g., in FIG. 2A, the septum-piercing means (230) is positioned in the centre of a spoke-wheel shaped part (220) positioned in the receiving device's inlet opening (210), i.e., the opening through which the dissolved or diluted drug formulation (110b) is received. In a more specific embodiment, the spoke-wheel shaped part (220) is shaped and/or positioned in the receiving device's inlet opening (210) in such a way that the piercing tip (231) of the septum-piercing means (230) is not jutting out, or protruding, from the surface of the outer casing (240) of the receiving device (200). This is beneficial insofar as there is a decreased injury risk for users as well as a reduced risk of materials other than skin getting caught on the piercing tip, either during use and/or during storage. In one embodiment (not depicted), the spoke-wheel shaped part (220) is shaped concavely, and the center, or hub, of said spoke-wheel shaped part (220) sits deeper within the inlet opening (210) than the wheel's outer circumference so as to protract the piercing tip (231) beneath the surface of the outer casing (240). Alternatively, as shown in e.g., FIG. 2A, the same tip-protracting effect could be achieved with a flat, or level, spoke-wheel shaped part (220; i.e., wheel hub and circumference in the same plane) by positioning the whole spoke-wheel shaped part (220) deeper within the inlet opening (210).

In one embodiment of the seal-type drug mixing system (10a), the septum-piercing means (230) is provided in the form of a septum-piercing cannula (230a), or hollow needle, or a so-called ‘spike’ (such as the Rowe Spike provided e.g., by Mediplast, or the Mini-Spike by B. Braun Melsungen). Depending on the properties of the septum (131), such as thickness, mechanical properties of the septum material, etc., the liquid expelled through the outlet port (130) upon piercing septum (131) may then pass either completely through said septum-piercing cannula/hollow needle/spike (230a), or—in cases where the cannula/needle creates a larger opening through the septum (131)—it may pass partially through the septum-piercing cannula/hollow needle (230a) and partially along the outsides thereof.

In an alternative embodiment, the septum-piercing means (230) is provided in the form of a solid septum-piercing pin (230b); i.e., without a bore or channel for fluid flow through its center. Depending on the properties of the septum (131), the septum-piercing pin (230b) may either puncture a hole into the septum (131), or open and widen a pre-existing opening, such as a tightly sealed slit through the septum (131).

Optionally, the septum-piercing means (230) can be provided with structural features on its outer perimeter which cause the opening pierced into the septum (131) to increasingly widen the further the septum-piercing means (230) is inserted; for instance, the outer perimeter of septum-piercing means (230) may be equipped with a set of spike-ribs (232) that widen from the tip towards the stem of said septum-piercing means (230). This can be beneficial for the speed and completeness of emptying the vial (110) contents, i.e., the dissolved or diluted drug formulation (110b), through the outlet port (130) and into the receiving device (200).

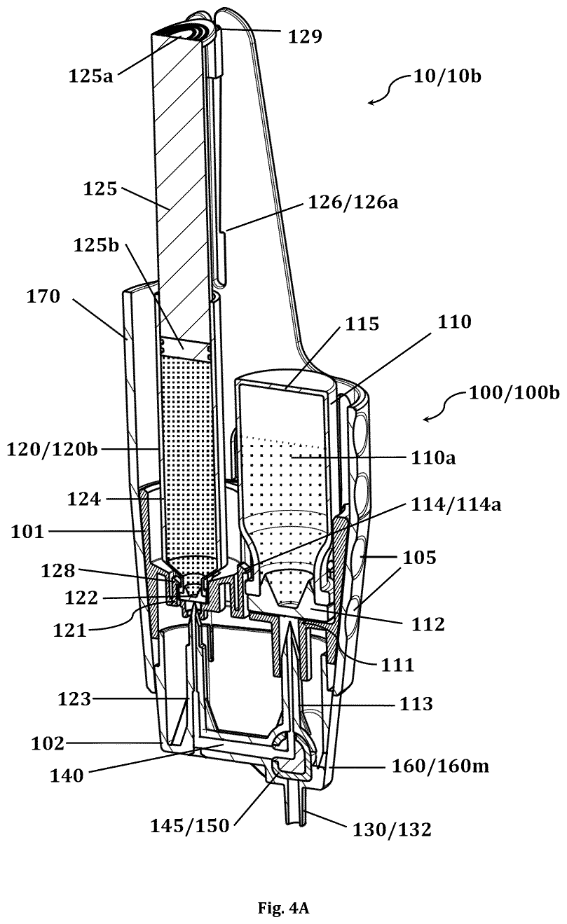

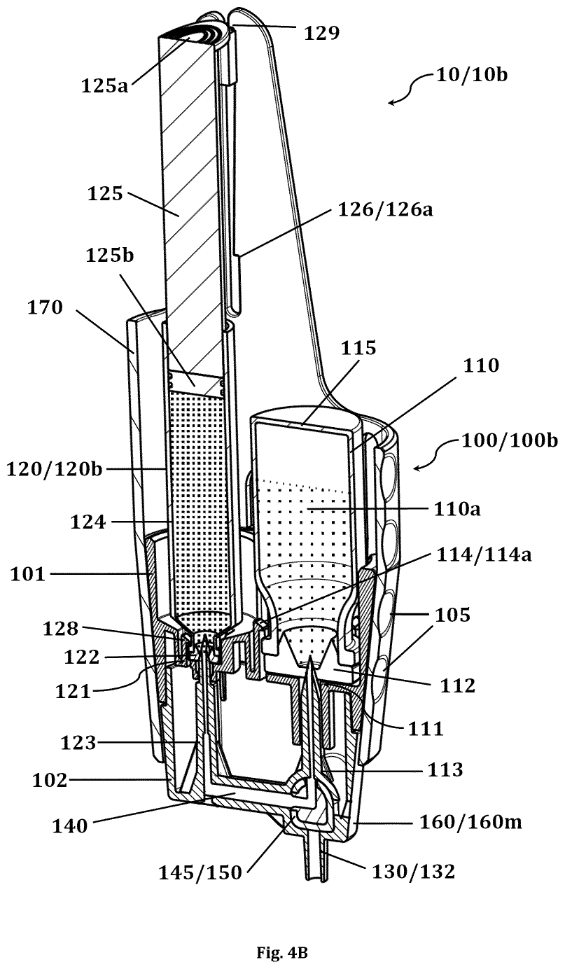

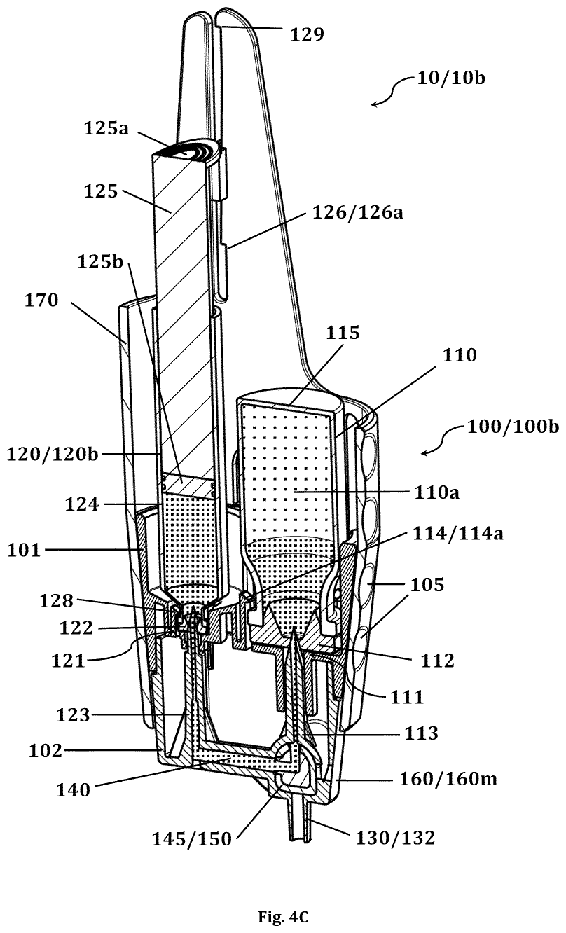

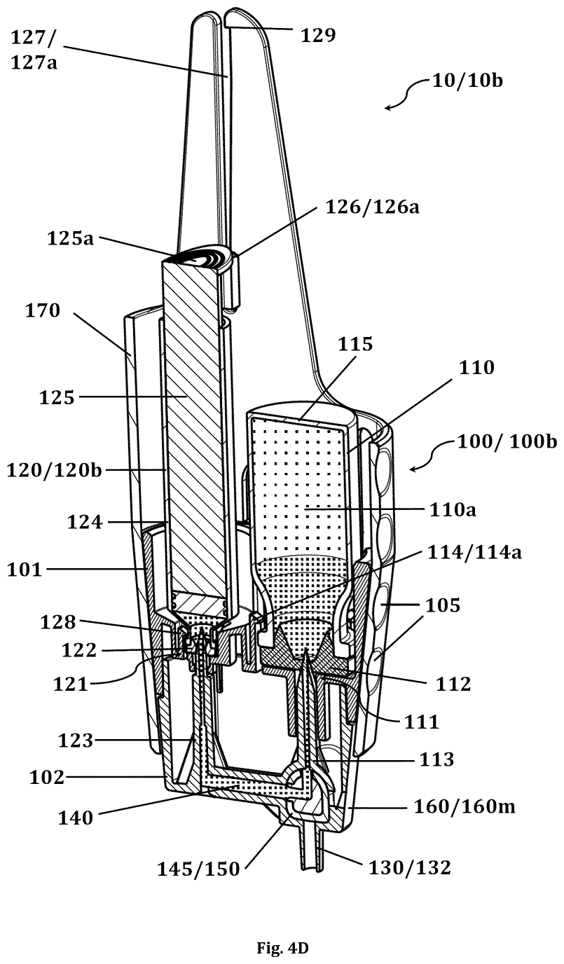

As further mentioned above, the drug mixing system (10) according to the first aspect of the invention, and more specifically the adapter (100) thereof, is equipped with a piston locking means (126) adapted to lock the position of the piston (125) once a predetermined volume of the liquid has been transferred from the reservoir (120) into the vial (110) by means of pressing the piston (125) into the housing (124). As can be seen in the figures, this locking of the piston (125) is achieved by a physical interaction of the piston head (125a) with the adapter's piston locking means (100); or in other words, the piston (125) is locked via its head end (125a). In one embodiment, as depicted e.g., in FIG. 1A-1E, FIG. 4A-4D, FIG. 9A-D, or FIG. 10A-D, the piston locking means (126) is provided in the form of an engagement hook (126a), or a plurality thereof (126a, 126a′, 126a″ etc.), a ratchet mechanism, or a similar snap-fit locking mechanism (126c), or a plurality thereof (126c, 126c′, 126c″ etc.). The adapter's piston locking means (126) interacts physically with the piston (125), and more specifically with the piston head (125a); for instance, with an engagement hook (126a) that traps the piston, and locks it in position, by hooking over the upper surface of the piston head (where a user would place their finger when pressing the piston down into the reservoir housing (124)) and thereby prevents the piston from travelling back, out of the housing, under the force of the overpressure when the user removes their finger from the piston head.

To facilitate the piston's insertion into the housing (124) but prevent a backwards motion of the piston (125) once inserted past the position of an engagement hook (126a), the latter is preferably adapted with a down-ward slant only in the direction of piston insertion but a flat, unslanted surface in the opposite direction (see e.g., FIG. 9D).

Alternatively, instead of hooking over the piston head's upper surface, a resilient, springy tailhook (126b) can be provided underneath the piston head's upper surface (as depicted exemplarily in FIG. 9A-9D), said tailhook (126b) being fixedly associated with, or forming an integral part of, the piston head (125a) and being adapted to be trapped under the engagement hook (126a), or a plurality thereof (126a, 126a′, 126a″ etc.). As shown in more detail in FIG. 9D, the piston head's tailhook (126b) springs into one of the three depicted engagement hooks (126a, 126a′, 126a″) once the piston (125) is inserted deep enough.

With respect to the expression, an engagement hook (126a), or a plurality thereof (126a, 126a′, 126a″ etc.), or a snap-fit locking mechanism (126c), or a plurality thereof (126c, 126c′, 126c″ etc.), it should be understood that these pluralities of engagement hooks or snap-fit locking means are intended to allow for a graduated insertion and locking of the piston (125) in more than one step; for instance, in FIG. 9A-9D in three steps (transferring ⅓, ⅔, or 3/3 of the liquid housed in the reservoir into the vial), and in FIG. 10A-10D in two steps (transfer ½ or 2/2 of the liquid).

Irrespective, of whether there is only one or more insertion steps for the piston (125), the drug mixing system (10), or more specifically its adapter (100), is configured such that the user will notice each insertion step by a tactile and/or an acoustic feedback; e.g. a small ‘click’ sensation and/or sound. In addition, the respective insertion depths for the piston (125) may also be marked on the outside of the adapter's stabilising wall (170) by visible markings (173); for instance, in the form of raised markings, as shown in FIGS. 9A and 9B. Alternatively, these markings (173) could be engraved or printed.

The benefit of providing a plurality of engagement hooks (126a, 126a′, 126a″ etc.) or a plurality of snap-fit locking means (126c, 126c′, 126c″ etc.) is that-without changes being required to the reservoir's housing (124), often a standardised container such as a syringe or pharmaceutical cartridge (120b)—different volumes of liquid (120a) can be transferred to the vial (110) and thus different corresponding overpressures generated in response. Provided that the solubility and/or viscosity parameters of the drug formulation (110b) formed permit it, a user would thus be enabled to, for instance, inhale a specific drug dose stored in the vial (110) in a shorter time, such as half the time if only half the volume of liquid (120a) housed in the reservoir (120) was used. The drug mixing system (10) according to the invention thereby allows for more flexibility in the preparation of the dissolved or diluted drug formulation (110b) than prior art devices.

In one embodiment, the engagement hook or hooks (126a, 126a′, 126a″ etc.) are shaped at/in a piston guiding groove (127a) formed in or through the adapter's stabilising wall (170), as depicted e.g., in FIGS. 1, 3, 4, and 6-8. In an alternative embodiment, the engagement hook or hooks (126a, 126a′, 126a″) jut out from a piston guide rail (127aa) formed on, or protruding from, an inner surface of the adapter's stabilising wall (170), as depicted in FIG. 9A-9D. More information on the piston guiding means (127) is provided further below.

In one embodiment, the piston locking means (126) is provided in the form of a snap-fit locking mechanism (126c), or a plurality thereof (126c, 126c′, 126c″ etc.), for instance, as one or more through-hole(s) in the adapter's stabilising wall (170), such as the first and second through-holes (126c, 126c′) exemplarily depicted in FIG. 10A-10D. The two through-holes are placed such as to transfer either one half or both halves of the liquid (120a) from the reservoir into the vial, and thus generating a corresponding overpressure. For the piston (125), or more specifically the piston head (125a) to get caught, or locked, at the position of the one or more through-hole(s), it is equipped at the piston head (125a) with a corresponding piston locking protrusion (126d) of appropriate size and shape to ‘snap-fit’ into the through-hole(s), when reaching their position upon insertion of the piston (125); see e.g., top of FIG. 10C.

The dimensions and mechanical strength of the stabilising wall (170) as well as the shape of the through-hole(s) are adapted such that at no point the generated overpressure would be sufficient to overcome the structural resistance of the through-hole(s) when the user removes their finger from the piston head (125a), meaning that the piston (125) could not unintentionally become unlocked and slide back out of the housing (124). At the same time, they are adapted such that in order to push the piston (125), for instance, from a first through-hole (126c) to a second through-hole (126c′), the user can overcome the structural resistance of the first ‘snap-fit’ simply by briefly exerting slightly more pressure on the piston head (125a).

The snap-fit of the piston locking protrusion (126d) into the one or more through-hole(s) (126c, 126c′, 126c″) in the adapter's stabilising wall (170) is noticeable via a tactile and/or an acoustic feedback; e.g., a small ‘click’ sensation and/or sound. In addition, the piston locking protrusion (126d) at the piston head (125a) may also be of a different colour than the material of the stabilising wall (170) so as to show visually once snap-fitted into one of the two through-holes (126c, 126c′), as shown in FIG. 10C. The provision of two through-holes is meant to allow for a graduated piston insertion and locking in half-steps, i.e., allowing the transfer of one half or both halves of the liquid (120a) housed in the reservoir (120) into the vial (110).

In addition to the above-described pluralities of piston locking means (126, 126′, 126″), which are aimed at allowing a graduated piston insertion, the adapter (100) of the drug mixing system (10) according to the first aspect of the invention may also be equipped with multiple sets of piston locking means (126), such as two sets thereof, with one being provided on either side of the adapter (100) and the piston head (125a) (i.e., spaced 180° apart), as visible e.g., in FIG. 1D, FIG. 3A, FIG. 7A, or FIG. 9D. This feature provides for added structural stability, allowing the piston (125) to be securely locked into position even at high overpressures generated.

To aid steady piston insertion even at high overpressures generated, there may also be multiple sets, e.g., two sets of piston guiding means (127/127a/127b), in addition to the multiple sets, e.g., two sets of the piston locking means (126, 126a, 126a′, 126a″, 126b), as also visible e.g., in FIG. 1D, FIG. 3A, FIG. 7A, or FIG. 9D.

In addition to the piston locking means (126), the drug mixing system (10) according to the first aspect of the invention may further comprise a piston securing means (129) which prevents loss, disconnection, or removal, of the piston (125) from the reservoir's housing (124); also see e.g., in FIG. 1A-1E, FIG. 4A-4D, or Fig, 9A-9D. As can be further seen in these figures, the piston securing means (129) may also be provided in multiple sets, e.g., two sets of piston securing means (129).

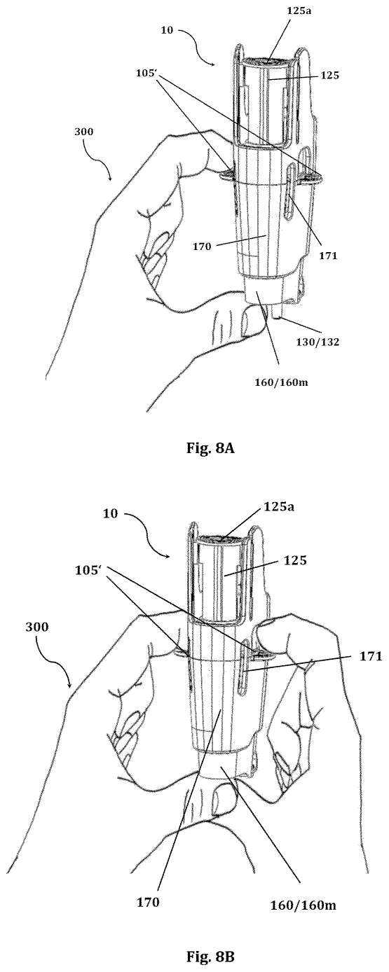

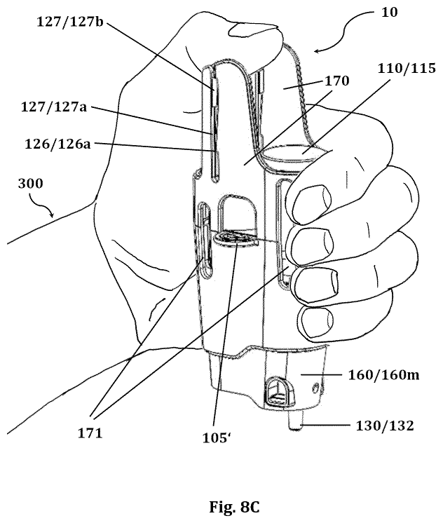

Moreover, the adapter (100) of the drug mixing system (10) according to the first aspect of the invention may further be equipped with a stabilising wall (170) which externally surrounds, or encases, at least parts of the reservoir housing (124), said stabilising wall (170) being dimensionally stable so as to support and stabilise the reservoir (120) within the adapter (100) while pressing the piston (125) into the housing (124) and generating the overpressure. Preferably, the stabilising wall (170) also externally surrounds, or encases, at least parts of the vial (110) so as to provide even better stability of the drug mixing system (10); for instance, to prevent that the vial (110) and/or reservoir (120) become unintentionally dislodged from their respective mounting openings (111, 121) when the user tightly grabs the drug mixing system (10) in one hand (300), as depicted e.g., in FIG. 8C, while pressing down the piston (125) with the thumb of the same hand (300). In that regard, the expression that the stabilising wall (170) externally surrounds, or encases, “at least parts of” the reservoir housing (124) and optionally of the vial (110), is to be understood in such a way that at least a stabilizing fraction of the outer surface of the reservoir housing (124), and optionally of the vial (110), is protected against dislodging by the stabilising wall (170); for instance, that those parts thereof are covered where, during use, the fingers of the hand (300) that is holding the drug mixing system (10) and pressing down the piston (125) would typically create pressure against the reservoir housing (124) and optionally of the vial (110).

In one embodiment, the stabilising wall (170) furthermore externally surrounds, or encases, at least parts of the adapter (100), and in particular at least the first subunit (101) thereof, in a sleeve-like manner. In other words, the stabilising wall (170) part is provided separately from, yet firmly connected to, the adapter (100). This is beneficial insofar as the stabilising wall (170) helps to guide the movement of the first and second adapter subunits (101, 102) towards each other during the activation step of the adapter (100), and it reduces the risk of the two adapter subunits (101, 102) getting dislodged from one another; for instance, upon insertion of the piston (125) into the reservoir's housing (124), where the user's hand (300) exerts force onto the drug mixing system (10), as depicted e.g., in FIG. 8C.

In an alternative embodiment, the stabilising wall (170) is formed as an integral part of the first adapter subunit (101).

Preferably, at least parts of the stabilising wall (170) are provided as a viewing window (171), for instance, by the stabilising wall (170) being formed from a see-through wall material and/or by providing cut-outs in a wall material; as depicted e.g., in FIG. 6A. The provision of the viewing window (171) is preferred insofar as it allows the user to check if the mixture of the drug formulation (110a) stored in the vial (110) and the liquid transferred from the reservoir (120) into the vial (110) is dissolved or diluted to a desired extent, prior to actuating the liquid-releasing means (155, 255), or whether either allowing further time and/or shaking of the drug mixing system (10) is needed for sufficient dissolution or dilution. In that regard, it should be understood that, typically, the expression desired extent of dissolution or dilution refers a homogeneous solution or, as the case may be, a homogeneous suspension or a homogeneous emulsion, with the homogeneity being understood as being evaluated with the naked human eye. However, ultimately, the ‘desired extent’ is something that is defined by the provider of the drug product (i.e., the drug formulation (110a) stored inside the vial (110)) and that can be found in the product leaflet or similar use information thereof. Thus, even if a homogenous solution could-in a theoretical example-be obtained from a homogenous suspension over an expanded dissolution time, but the leaflet or use information calls for application of the suspension, then the homogenous suspension is what is to be considered the desired extent of mixing.

In one embodiment, the piston locking means (126) is formed as an integral part of the stabilising wall (170), as depicted e.g., in FIG. 1A-1E, FIG. 4A-4D, or FIG. 9A-9AD. This is beneficial insofar as it facilitates the casting, or injection moulding process (i.e., the piston locking means can be cast together with the stabilising wall) and provides a simple, yet dimensionally stable option to lock the piston (125) in place once a predetermined volume of liquid has been transferred from the reservoir (120) over into the vial (110). In a specific embodiment, both the piston locking means (126) and the optional piston securing means (129) are formed as integral parts of the stabilising wall (170), also see e.g., in FIG. 1A-1E, FIG. 4A-4D, or FIG. 9A-9D.

In one of the preferred embodiments, the adapter (100) is further equipped with a piston guiding means (127) that enables a controlled, steady movement of the piston (125) within the housing (124) while pressing the piston (125) into the housing (124) and generating the overpressure. As depicted in e.g., FIGS. 3A and 3B or FIGS. 9A and 9B, respectively, this piston guiding means (127) may, for instance, be provided (i) in the form of a piston guiding groove (127a), and a corresponding protrusion (127b) on the piston (125), more specifically on the piston head (125a), which slides down the groove (127a) while pressing the piston (125) into the housing (124) and generating the overpressure; or (ii) in the form of a piston guide rail (127aa) protruding from an inner surface of the stabilising wall (170), and a corresponding notch (127bb) on the piston (125), or its piston head (125a), such as an I-beam shaped guide rail and a corresponding ‘double-T’ notch which slides down the piston guide rail (127aa) while pressing the piston (125) into the housing (124) and generating the overpressure. Depending on the properties of the stabilising wall (170), such as its thickness and/or hardness, the piston guiding groove (127a) may be provided either in an inner surface of the stabilising wall (170; i.e., the surface facing the reservoir); or it may reach all the way through the stabilising wall (170) in the form of a cut-out (as shown e.g., in FIGS. 1, 3, 4, 6 and 7). To support and stabilise the piston movement further, the corresponding piston protrusion (127b) sliding through said guiding groove (127a) may be provided with flanges (127b′, 127b″; see e.g., FIG. 7A-C) that are contacting the inner surface, or optionally both the inner-and outer surfaces of the stabilising wall (170). Where a piston guide rail (127aa) type of piston guiding means (127) is chosen, this can optionally be provided as an integral part of the stabilising wall (170), e.g., cast, or injection moulded, together with said wall.

As mentioned above, the adapter (100) of the drug mixing system (10) may be equipped with multiple sets of piston guiding means (127/127a/127b), such as two sets thereof, with one being provided on either side of the adapter (100) and the piston head (125a) (i.e., spaced 180° apart), as visible e.g., in FIG. 1D, FIG. 3A, FIG. 7A, or FIG. 9D. This provides for added structural stability and facilitates steady piston insertion, less prone to rotational dislocation, even at high overpressures.

Since the drug mixing system (10) according to the first aspect of the invention is meant to be operable single-handedly (i.e., holding the system in one hand (300) and using the thumb of said hand (300) to press down the piston (125)), in one of the preferred embodiments, the vial-mounting opening (111) is equipped with a vial fixation means (114) for unreleasably connecting the vial (110) to the vial-mounting opening (111). Similarly, in another preferred embodiment, the reservoir-mounting opening (121) is equipped with a reservoir fixation means (128) for unreleasably connecting the reservoir (120) to the reservoir-mounting opening (121). In one embodiment, both the vial-and the reservoir-mounting openings (111, 121) are equipped with respective vial-and reservoir fixation means (114, 128) for unreleasably connecting the vial (110) and the reservoir (120) to their respective mounting openings. This is not only beneficial for the handling stability of the drug mixing system (10) during use but may -in the more common case of prefilled and packaged drug mixing systems (10) with the vial (110) and reservoir (120) pre-assembled in the adapter (100)—also serve as a means to prevent misuse, unintended multiple-use, or to prevent accidental mispairings of e.g., a drug formulation (110a) with a reservoir of the wrong liquid for dissolution.

As mentioned above, the drug mixing system (10) according to the first aspect of the invention comprises a reservoir (120) with a housing (124) and a piston (125) sealingly movable within said housing (124). In one embodiment, the reservoir (120) is provided in the form of a syringe (120c), optionally a syringe equipped with a LUER-slip without a thread (LUER slip) or with a LUER-lock with a thread (LUER lock). Preferably, the syringe (120c), or at least its housing (124) is a standard syringe (housing) as commercially available, and does not require, for instance, the provision of any customized openings to be suited for the use with the drug mixing system (10) of the present invention. In an alternative embodiment, the reservoir (120) is a pharmaceutical cartridge (120b). These cartridges (120b) are also commercially available, e.g., in standardized volumes of 1.5 mL, 3.0 mL or 5.0 mL. Overall, they are similar to standard syringes, with some of the differences being that (i) pharmaceutical cartridges (120b) are usually made of glass, not plastic like syringes, and that (ii) unlike most syringes, cartridges are commonly sealed at their dispensing tip, or ‘downstream’ end; e.g., by a crimped-on stopper meant to be pierced open during use. The opposite wider end, or ‘upstream’ end is usually sealed for both syringes and cartridges-at least for prefilled systems-by parts of the piston (125), with usually at least the sealing gasket, or stopper (125b), of the piston (125), or plunger, being inserted into the housing (124).

Typically, the vial (110) of the drug mixing system (10) according to the first aspect of the invention is sealed with a pierceable vial lid (112), and the adapter (100) comprises a respective vial-piercing cannula (113) adapted to pierce the vial lid (112) and access the drug formulation (110a) in the vial (110) during use of the drug mixing system (10). One of the most common pierceable lid types used for commercially available, and in particular for prefilled, vials is a stopper-made of rubber or a similarly sealing elastomeric material as mentioned above-crimped around the neck of the vial (110) with a metal closure, or alternatively affixed by heat-staking. This offers the best protection of the drug formulation (110a) in the vial (110) from e.g., humidity, oxygen, or the like. With respect to the vial-piercing cannula (113) it should be understood that this cannula (113) is not identical to, or the same as, the liquid-release means (155, 255), e.g., the septum-piercing means (230), or more specifically the septum-piercing means in form of a cannula/hollow needle/spike (230a). The vial-piercing cannula (113) is only adapted to pierce the vial lid (112) and allow (i) for liquid from the reservoir (120) to enter the vial (110) and (ii) for the mixture formed from said liquid and the drug formulation (110a) to exit the vial (110), and flow into the internal adapter channel (140), under the increasing overpressure generated in the vial (100).

In one of the preferred embodiments, the vial-piercing cannula (113) is positioned in vertical alignment between the vial (110) and the outlet port (130) and its circumferential wall (132), rather than at an angle thereto (i.e., their respective longitudinal central axes falling in line, as exemplarily depicted in FIG. 1E, or their longitudinal central axes being at least parallel to each other and only a short distance of no more than 5 mm, preferably no more than 2 mm off-set from one another) to allow for unhindered, overpressure-driven, gravity-assisted, non-sinuous flow from the vial (110) towards the outlet port (130). For the same reason, it is further preferred that the vial-piercing cannula (113) is straight, non-collapsible, and non-tortuous. Further preferably, the vial-piercing cannula (113) is not longer than 20 mm, or not longer than 15 mm, or not longer than 10 mm. In one embodiment, the vial-piercing cannula (113) exhibits a length of 5-20 mm, of 5-15 mm, or of 5-10 mm.

It is further preferred, that the distance between the vial (110) and the outlet port (130), as measured between the upstream end of the vial mounting opening (111) (i.e., the surface, or plane, on which the vial (110) rests), and the downstream end of the outlet port (130), or its circumferential wall (132) (i.e., where the dissolved or diluted drug formulation (110b) exits from the adapter), is not longer than 40 mm, or not longer than 35 mm, or not longer than 30 mm. In one embodiment, the distance between the vial (110) and the outlet port (130) is in the range of 25-40 mm, of 25-35 mm, or of 25-30 mm. In one embodiment, the distance between the vial (110) and the outlet port (130) is not more than 20 mm longer than the length of the vial-piercing cannula (113), or not more than 15 mm, not more than 10 mm.

The above is advantageous insofar that both windy, tortuous flow-paths between the vial (110) and the outlet port (130) and those longer than described above, for instance, use of curved or bent flexible tubes in the adapter (100), are detrimental for expelling the dissolved or diluted drug formulation (110b) from the adapter or transferring it into the receiving device (200) with the use of overpressure. This applies in particular when either (i) the diluted drug formulation (110b) is not a solution but could be an emulsion or a suspension instead, and/or when (ii) the amount of liquid (120a) transferred from the reservoir (120) into the vial (110), and consequently the overpressure in the vial (110) resulting from it, is meant to be dosable (e.g., dosable in half-steps, or in thirds), and may not always use ‘full-force’ for expulsion from the adapter (100). If curved or bent tubing is used, optionally flexible tubes, such as when the outlet port is provided at a 90° angle to the vial, suspended particles in a diluted drug formulation (110b) are at higher risk of, sedimenting in the curves/bends when the overpressure is lower, or they may get pressed and caked together in the curves/bends when the overpressure is high. This could lead to drug losses, inaccurate dosing, as well as to the mixing adapter getting clogged and unusable altogether. The drug mixing system (10) of the present invention helps prevent these drug losses and dosing inaccuracies.

In one embodiment, the reservoir (120) is also sealed with a reservoir lid (122) adapted to open during use of the drug mixing system (10) so as to access the liquid in the reservoir (120). This could either be a pierceable lid or another type of closure that opens and releases the liquid from the reservoir (120) when the piston (125) is pressed into the housing (124); for instance, a slit-or duckbill-valve, or a membrane with one or more slits which open(s) under pressure. In a specific embodiment, the reservoir (120), much like the vial (110), is sealed with a pierceable reservoir lid (122), and the adapter (100) further comprises a respective reservoir-piercing cannula (123) adapted to pierce, or puncture, the reservoir lid (122) and access the liquid in the reservoir (120) during use of the drug mixing system (10). For instance, as mentioned above, the reservoir (120) could be a pharmaceutical cartridge (120b) with a stopper, typically of rubber or a similarly sealing elastomeric material, crimped, or otherwise tightly affixed onto its dispensing tip, or ‘downstream’ end, that is meant to be pierced open during use.

In one embodiment, the adapter (100) comprises a first adapter subunit (101), and a second adapter subunit (102), wherein the first and second adapter subunits (101, 102) are connected with one another and engageable in such a way that the adapter (100) has a resting position and an activated position. The resting position is the position in which the vial-piercing cannula (113) does not yet pierce the vial lid (112); or, as the case may be, the vial-and reservoir-piercing cannulae (113, 123) do not yet pierce the respective vial-and reservoir lids (112, 122). The activated position is the position in which, during use of the drug mixing system (10), the first and the second adapter subunits (101, 102) are shifted towards each other and thereby brought into a position in which the vial cannula (113) pierces the vial lid (112) thereby accessing the drug formulation (110a) in the vial (110); or, as the case may be, the vial-and reservoir-piercing cannulae (113, 123) pierce the respective vial-and reservoir lids (112, 122), thereby accessing the drug formulation (110a) in the vial (110) and the liquid in the reservoir (120). The provision of the two subunits (101, 102) of the adapter (110), and thus of the two positions ‘resting’ vs. ‘activated’, is beneficial insofar as the vial contents, optionally both the vial-and the reservoir contents, are protected from e.g., humidity, oxygen, etc. when the drug mixing system (10) is not yet in use. In other words, in one embodiment, prior to use (such as for shipping, delivery, and storage), the adapter (100) is provided in its resting position.

In one embodiment, the drug mixing systems 10), or the adapter (100) thereof, comprise an indicator window (172), as depicted e.g., in FIG. 9A-9C or FIG. 10B, which indicates to the user, typically in a visual manner, whether the adapter (100) is in its activated position (i.e., whether the two adapter subunits have been moved towards each other and thus the vial and reservoir opened/pierced). The indicator window (172) could turn green, for instance, once the adapter (100) is activated. Alternatively, or in addition, the drug mixing systems 10), or the adapter (100) thereof, could comprise a tactile activation indicator instead of a colour-changing one; such as a knob that springs out through the indicator window (172) once the adapter (100) is brought into its activated position.

Commonly, the adapter (100) is provided to the user in pre-assembled form, for instance, with the first and second adapter subunits (101, 102) of the adapter (100) being pre-assembled. In one of the preferred embodiments, the first and second adapter subunits (101, 102) are unreleasably connected when in the resting position. This prevents the two subunits coming apart unintentionally prior to use, upon unpacking the drug mixing system (10) and/or when moving the adapter (100) from resting into activated position. In a further preferred embodiment, the first and second adapter subunits (101, 102) are unreleasably connected when in the activated position. This prevents two subunits coming apart unintentionally during use (e.g., when pressing the piston (125) into the reservoir housing (124)), and thus spillage or contamination of the vial and/or reservoir contents.

In order to allow for these unreleasable connections between the first and second adapter subunits (101, 102), in one embodiment, the first and/or the second adapter subunit (101, 102) comprise at least one adapter subunit locking means (103) for the locking engagement of the first adapter subunit (101) with the second adapter subunit (102). In a specific embodiment, the adapter subunit locking means (103) is adapted to unreleasably lock the adapter (100) in the resting position or in the activated position. In a more specific embodiment, the adapter subunit locking means (103) is provided in the form of one or more subunit locking hook(s) (103a) and corresponding subunit locking groove(s) (103b) positioned inside the first and second adapter subunits (101, 102). In a further specific embodiment, the first and/or the second adapter subunit (101, 102) comprise at least two adapter subunit locking means (103) for the engagement of the first adapter subunit (101) with the second adapter subunit (102). In a yet further specific embodiment, one of the at least two adapter subunit locking means (103) is engaged in the resting position, and the other one is engaged in the activated position.

For bringing the adapter (100) from its resting into its activated position, the two adapter subunits (101, 102) are typically shifted towards each other, or pressed or ‘clicked’ together; for instance, by holding the drug mixing system (10) in one hand (300), as depicted e.g., in FIG. 8A or FIG. 10B (or between two hands (300) as depicted in FIG. 8B), with the thumb(s) resting against the flat bottom end, or lower end, of the adapter (100) and with the fingers pressing onto either the bottom (115) of the vial (110), or, preferably, onto one or more dedicated sites shaped as patterned ledge(s) (105′) for the placement of fingers, said ledges jutting out, for instance, from the stabilising wall (170); e.g., one on either side of the drug mixing system. Not pressing onto the bottom (115) of the vial (110) but instead onto dedicated sites shaped as patterned ledge(s) (105′) for the placement of fingers is preferred insofar as it puts less strain onto the vial fixation and avoids dislodging of the vial (110).

In one embodiment, the first and second adapter subunits (101, 102) are adapted such that they can be shifted towards each other single-handedly. Therefore, in one of the preferred embodiments, the adapter (100), preferably the second adapter subunit (102), comprises at least one dedicated site for resting one or more finger(s) (105, 105′) when bringing the adapter (100) from the resting position into the activated position. This facilitates holding and using the adapter (100) single-handedly as it limits the risk of the finger(s) slipping. The dedicated site (105, 105′) can, for instance, be an approximately finger-tip sized recess, or indentations placed at the lower end of the second adapter subunit (102) such as a wing-shaped form, or curve, to comfortably rest one or two fingers in the concave part(s) of said form. Alternatively, and as depicted in e.g., FIG. 7A-7C or FIG. 8A-8C, the lower end of the second adapter subunit (102) can be provided as a simple flat surface against which e.g., an opposing thumb could rest, and the dedicated site(s) can be provided in the form of a patterned ledge (105′) extending at an approx. 90° angle on at least one side, preferably both sides, of the drug mixing system (10; e.g., extending from the stabilising wall (170)) so as to rest one at least one finger during use, and especially while bringing the adapter (100) from its resting into its activated position. Alternatively, to the finger placing shown in FIG. 8A, the user could also place the drug mixing system (10) between the splayed index-and middle finger during the adapter (100) activation step.

In one embodiment, the first adapter subunit (101) comprises at least a vial-mounting opening (111) adapted to hold the vial (110), a reservoir-mounting opening (121) adapted to hold the reservoir (120), and optionally the stabilising wall (170) which externally surrounds, or encases, at least parts of the reservoir's housing (124). Or, in other words, the vial-mounting opening (111), reservoir-mounting opening (121) and optionally the stabilising wall (170) of the adapter (100) are part(s) of the of the adapter's first subunit (101). Where the stabilising wall (170) is present, as mentioned above, it can be formed as an integral part of the first adapter subunit (101).

In one embodiment, the second adapter subunit (102) comprises at least the switch valve (150) and the outlet port (130); or, in other words, the switch valve (150) and the outlet port (130) of the adapter (100) are part(s) of the of the adapter's second subunit (102).

As mentioned, in order to bring the adapter (100) from its resting into its activated position, the two adapter subunits (101, 102) are typically shifted towards each other. To facilitate this motion, in one embodiment, the adapter (100) comprises adapter subunit guiding means for guiding the movement of the first adapter subunit (101) and the second adapter subunit (102) when shifting them towards each other from the resting position into the activated position. Similar to the piston guiding means (127), the adapter subunit guiding means, in one embodiment, is provided in the form of at least one subunit guiding recess in the first adapter subunit (101) and at least one corresponding subunit guiding protrusion fitting the recess in the second adapter subunit (102); or vice versa. In an embodiment, where the stabilising wall (170) externally surrounds, or encases, at least parts of the first and second adapter subunits (101, 102) in a sleeve-like manner, such as depicted e.g., in FIG. 1A-1E or in FIG. 4A-4D, the stabilising wall (170) may also act as an adapter subunit guiding means insofar as it guides the motion of the two subunits towards each other.

In one embodiment, the vial (110) of the drug mixing system (10) according to the first aspect of the invention exhibits a longitudinal central axis ‘A’ (as exemplarily depicted in FIG. 1E or FIG. 6B), and said longitudinal central axis ‘A’ is positioned vertical, or essentially vertical, during use, or more specifically while the dissolved or diluted drug formulation (110b) is expelled, or transferred, from the vial (110) through the outlet port (130). This is advantageous insofar as it helps to ensure complete and rapid emptying of the vial (110). Thus, in one of the preferred embodiments, the drug mixing system (10) and the receiving device (200) are adapted such that upon connecting them, this vertical, or essentially vertical, position of the vial (110), or its said longitudinal central axis ‘A’ during use is ensured. For instance, in one embodiment, the vial (110) exhibits a longitudinal central axis ‘A’ and the receiving device (200) exhibits a longitudinal central axis ‘C’, and the vial's longitudinal central axis ‘A’ is oriented perpendicular to the receiving device's longitudinal central axis ‘C’ when the drug mixing system (10) is connected to the receiving device (200), as depicted e.g., in FIG. 6B. The receiving device (200), e.g., a nebulizer (201) such as a vibrating mesh nebulizer (202), may, for instance, be placed with a flat backside thereof onto the table, with its inlet opening (210) facing upward, or towards the user; and the drug mixing system (10) can then be placed on top, and connected to the receiving device (200) via the connecting-and counter connection means (160, 260) in such a way that the vial's longitudinal central axis ‘A’ is positioned vertical, or essentially vertical.

In one embodiment, the vial (110) exhibits a longitudinal central axis ‘A’, and the reservoir (120) exhibits a longitudinal central axis ‘B’ (see e.g., FIG. 6B), and the vial-and reservoir mounting openings (111, 121) are arranged to hold the vial (110) and the reservoir (120) in a position in which longitudinal central axes ‘A’ and ‘B’ are positioned at an angle below 75°, preferably below 60°, more preferably below 50°, or even below 20°, such as 0°. In a specific embodiment, the longitudinal central axes ‘A’ and ‘B’ are positioned at an angle between 20° and 0°, preferably between 10° and 0°, more preferably between 5° and 0°, such as 0°. For instance, as exemplarily depicted in FIG. 1E or FIG. 6B, the two longitudinal central axes ‘A’ and ‘B’ can be positioned at an angle of approx. 0°, or in other words parallel to each other. In an alternative embodiment, the longitudinal central axes ‘A’ and ‘B’ are positioned at an angle between 70° and 20°, or between 60° and 30°, or between 50° and 40°, such as 45°.

In one embodiment, the vial-and reservoir mounting openings (111, 121) of the adapter (100) are arranged in such a way that at least the vial's longitudinal central axis ‘A’, optionally both the vial's and the reservoir's longitudinal central axes ‘A’ and ‘B’, is/are positioned vertical, or essentially vertical, during use, or more specifically while the dissolved or diluted drug formulation (110b) is expelled, or transferred, from the vial (110) through the outlet port (130).

In one embodiment, the adapter (100), and optionally the drug mixing system (10) as a whole, is sterilizable. In a specific embodiment, the adapter (100), and optionally the drug mixing system (10) as a whole, is sterilizable in packaged state. In a specific embodiment, the adapter (100), and optionally the drug mixing system (10) as a whole, is sterilizable with ethylene oxide and/or gamma irradiation. In one of the preferred embodiments, the adapter (100), and optionally the drug mixing system (10) as a whole, is stored and delivered in a pre-packaged, sterilized state (see e.g., package (400) in FIG. 10A). It is furthermore preferred, that the vial (110), and the reservoir (120) are pre-assembled within the respective the vial-and reservoir mounting openings (111, 121) of the adapter (100) of the drug mixing system (10).

In one embodiment, the drug mixing system (10) is a valve-type drug mixing system (10b) (i.e., liquid-retaining means (145) in the form of a switch valve (150)), and the adapter (100), and optionally the drug mixing system (10) as a whole, is stored and delivered in a pre-packaged state with the switch valve (150) of the adapter (100) being in valve position 1.

In one embodiment, the drug formulation (110a) in the vial (110) is provided in powder form, such as a powder for reconstitution, or as a liquid. For instance, in a specific embodiment, the drug formulation (110a) in the vial (110) is provided in a lyophilized form, i.e., a lyophilized powder. In an alternative embodiment, the drug formulation (110a) in the vial (110) is provided as a liquid, for instance, a concentrate and/or a liquid solubilisate of the drug. In one embodiment, the drug formulation (110a) in the vial (110) comprises at least one drug, optionally a combination of drugs.

In one embodiment, the liquid (120a) in the reservoir (120) is a pharmaceutically acceptable liquid. For instance, in a specific embodiment, the pharmaceutically acceptable liquid is an injectable liquid and/or an inhalable liquid, such as water for injection (aqua ad injectabilia).

### In One Embodiment, the Drug Mixing System (10) Is Intended for Single-use.

In a second aspect, the present invention relates to an adapter (100) for the drug mixing system (10) according to the first aspect of the invention. Accordingly, any embodiments, or specific or preferred embodiments, disclosed herein in connection with the drug mixing system (10) according to the first aspect of the invention, may be applied to the adapter (100) according to this second aspect of the invention; for instance, that, in one embodiment, the adapter (100) is equipped with a piston locking means (126) adapted to lock the position of the piston (125) once a predetermined volume of the liquid has been transferred from the reservoir (120) into the vial (110) by means of pressing the piston (125) into the housing (124), thereby creating and storing an overpressure; or that in one embodiment, the adapter (100) equipped with a stabilising wall (170) which externally surrounds, or encases, at least parts of the reservoir housing (124), said stabilising wall (170) being dimensionally stable so as to support and stabilise the reservoir (120) within the adapter (100) while pressing the piston (125) into the housing (124) and generating the overpressure.

In a third aspect, the present invention relates to a method of preparing a dissolved or diluted drug formulation (110b), optionally a drug solution, from a drug formulation (110a) stored in a vial (110), using the drug mixing system (10) according to the first aspect of the invention. Accordingly, any embodiments, or specific or preferred embodiments, disclosed herein in connection with the drug mixing system (10) according to the first aspect of the invention, may be applied to the preparation method according to this third aspect of the invention, including any operational steps described above for the drug mixing system (10) according to the first aspect of the invention, or its adapter (100) according to the second aspect of the invention.

More specifically, according the third aspect, the invention provides a method comprising the following subsequent steps:
a) providing a drug mixing system (10) according to the first aspect of the invention;
b) with the liquid-retaining means (145) blocking at least the flow of dissolved or diluted drug formulation (110b) through the outlet port (130), transferring a predetermined volume of the liquid from the reservoir (120) into the vial (110) by means of pressing the piston (125) into the housing (124), thereby creating an overpressure and a mixture of the drug formulation (110a) and the liquid;
c) locking the piston (125) in place with the piston locking means (126) once the predetermined volume of the liquid has been transferred from the reservoir (120) into the vial (110), thereby storing the overpressure created in step b;
d) allowing the mixture of the drug formulation (110a) and the liquid obtained from steps b) and c) to get dissolved or diluted to the desired extent; optionally aiding this dissolution or dilution process by shaking the drug mixing system (10);
e) actuating the liquid-releasing means (155, 255), thereby expelling, or transferring, the dissolved or diluted drug formulation (110b) from the vial (110) through the outlet port (130) using the overpressure generated and stored in steps b) and c) by pressing the piston (125) into the housing (124) and locking the piston (125) in place.

For instance, in on embodiment, the method comprises the following subsequent steps:
a) providing a valve-type drug mixing system (10b) according to the first aspect of the invention, with the liquid-retaining means (145) being provided in the form of a switch valve (150), and the internal adapter channel (140) and the switch valve (150) being adapted to at least allow selective fluid connection between the vial (110) and the reservoir (120) while blocking fluid connection to the outlet port (130) when in valve position 1, and between the vial (110) and the outlet port (130) while blocking fluid connection to the reservoir (120) when in valve position 2;
b) with the switch valve (150) being in valve position 1, or being brought to valve position 1, transferring a predetermined volume of the liquid from the reservoir (120) into the vial (110) by means of pressing the piston (125) into the housing (124), thereby creating an overpressure and a mixture of the drug formulation (110a) and the liquid;
c) locking the piston (125) in place with the piston locking means (126) once the predetermined volume of the liquid has been transferred from the reservoir (120) into the vial (110), thereby storing the overpressure created in step b;
d) allowing the mixture of the drug formulation (110a) and the liquid obtained from steps b) and c) to get dissolved or diluted to the desired extent; optionally aiding this dissolution or dilution process by shaking the drug mixing system (10);
e) bringing the switch valve (150) into valve position 2, thereby expelling, or transferring, the dissolved or diluted drug formulation (110b) is from the vial (110) through the outlet port (130) using the overpressure generated and stored in steps b) and c) by pressing the piston (125) into the housing (124) and locking the piston (125) in place.

In an alternative embodiment, the method comprises the following subsequent steps:
a) providing a seal-type drug mixing system (10a) according to the first aspect of the invention, with the liquid-retaining means (145) being provided in the form of a septum (131) covering the outlet port (130), and the liquid-releasing means (155, 255) in the form of a septum-piercing means (230);
b) with the septum (131) blocking at least the flow of dissolved or diluted drug formulation (110b) through the outlet port (130), transferring a predetermined volume of the liquid from the reservoir (120) into the vial (110) by means of pressing the piston (125) into the housing (124), thereby creating an overpressure and a mixture of the drug formulation (110a) and the liquid;
c) locking the piston (125) in place with the piston locking means (126) once the predetermined volume of the liquid has been transferred from the reservoir (120) into the vial (110), thereby storing the overpressure created in step b;
d) allowing the mixture of the drug formulation (110a) and the liquid obtained from steps b) and c) to get dissolved or diluted to the desired extent; optionally aiding this dissolution or dilution process by shaking the drug mixing system (10);
e) actuating the liquid-releasing means (155, 255) by piercing the septum (131) with the septum-piercing means (230), thereby expelling, or transferring, the dissolved or diluted drug formulation (110b) from the vial (110) through the outlet port (130) using the overpressure generated and stored in steps b) and c) by pressing the piston (125) into the housing (124) and locking the piston (125) in place.

In one embodiment of the method according to the third aspect of the invention, a drug mixing system (10) comprising the first and second adapter subunits (101, 102) as described above is provided in step a) and said drug mixing system (10) is activated by bringing its adapter (100) from the resting position into the activated position as described above. In a specific embodiment, the adapter (100) is brought from the resting position into the activated position by shifting the first and second adapter subunits (101, 102) of the adapter (100) towards each other. In a more specific embodiment, the first and second adapter subunits (101, 102) of the adapter (100) are, or at least can be, shifted towards each other single-handedly.

In one embodiment of the method according to the third aspect of the invention, the dissolved or diluted drug formulation (110b) is transferred from the vial (110) via the outlet port (130) into a receiving device (200) for receiving the dissolved or diluted drug formulation (110b) from the vial (110).

Preferably, in order to make the use of the drug mixing system (10) as simple and convenient as possible for the user, the drug mixing system (10) is provided to the user with all components being together, i.e., with the adapter (100), the vial (110) housing the drug formulation (110a), and the reservoir (120) for housing the liquid (120a) for dissolution or dilution of the drug formulation (110a) being combined (e.g., in the same primary packaging (400)); and further preferably with the vial (110) and the reservoir (120) being preassembled in their respective mounting openings (111, 121). However, in embodiments, where the adapter (100) is provided separately from the vial (110) housing the drug formulation (110a), and/or the reservoir (120) for housing a liquid for dissolution or dilution of the drug formulation (110a), the vial (110) and/or the reservoir (120) are then coupled to the respective vial-and reservoir mounting openings (111, 121) of the adapter (100) so as to provide the drug mixing system (10) of method step a).

Furthermore, the inventive drug mixing and injection systems are material-saving as they use a reduced number of vials which reduces the consumption of disposable material.

### Item List

1. A drug mixing system (10) for dissolving or diluting a drug formulation (110a) stored in a vial (110) prior to use, the drug mixing system (10) comprising an adapter (100) adapted to hold the vial (110) housing the drug formulation (110a), and a reservoir (120) for housing a liquid (120a) for dissolution or dilution of the drug formulation (110a), the reservoir (120) comprising a housing (124) and a piston (125) sealingly movable within the housing,
   wherein the adapter (100) comprises:
   a vial mounting opening (111) adapted to hold the vial (110),
   a reservoir mounting opening (121) adapted to hold the reservoir (120),
   an outlet port (130),
   an internal adapter channel (140) fluidly connecting the vial-and reservoir mounting openings (111, 121), and the outlet port (130), and
   a liquid-retaining means (145) adapted to block at least a flow of dissolved or diluted drug formulation (110b) through the outlet port (130),
   characterized in that:
      the adapter (100) is equipped with a piston locking means (126) adapted to lock the position of the piston (125) once a predetermined volume of the liquid has been transferred from the reservoir (120) into the vial (110) by means of pressing the piston (125) into the housing (124), thereby creating and storing an overpressure,
      the drug mixing system (10) is adapted to expel, or transfer, the dissolved or diluted drug formulation (110b) from the vial (110) through the outlet port (130) upon actuation of a liquid-releasing means (155, 255) using the overpressure generated by pressing the piston (125) into the housing (124).
2. The drug mixing system (10) according to item 1, wherein
   a) for a seal-type drug mixing system (10a), the liquid-retaining means (145) is provided in the form of a septum (131) covering the outlet port (130), and the liquid-releasing means (155, 255) is provided in the form of a septum-piercing means (230) so that, upon actuation, the septum (131) is pierced by the septum-piercing means (230) and the dissolved or diluted drug formulation (110b) is expelled, or transferred, from the vial (110) through the outlet port (130) using the over-pressure generated by pressing the piston (125) into the housing (124); or
   b) for a valve-type drug mixing system (10b), the liquid-retaining means (145) is provided in the form of a switch valve (150), wherein the internal adapter channel (140) and the switch valve (150) are adapted to at least allow selective fluid connection between the vial (110) and the reservoir (120) while blocking fluid connection to the outlet port (130) when in valve position 1, and between the vial (110) and the outlet port (130) while blocking fluid connection to the reservoir (120) when in valve position 2; and
      the liquid-releasing means (155, 255) is provided in the form of a switch-valve operating means (156) adapted to, upon actuation, bring the switch valve (150) into valve position 2, so that, upon the switch valve (150) being brought into said valve position 2, the dissolved or diluted drug formulation (110b) is expelled, or transferred, from the vial (110) through the outlet port (130) using the over-pressure generated by pressing the piston (125) into the housing (124).
3. The drug mixing system (10) according to items 1 or 2, wherein the dissolved or diluted drug formulation (110b) is transferred from the vial (110) via the outlet port (130) into a receiving device (200) for receiving the dissolved or diluted drug formulation (110b) from the vial (110).
4. The drug mixing system (10) according to item 3, wherein the receiving device (200) is a nebulizer (201).
5. The drug mixing system (10) according to items 3 or 4, wherein the receiving device (200) is a vibrating mesh nebulizer (202), and wherein the dissolved or diluted drug formulation (110b) from the vial (110) is transferred into, or received by, a nebulizing chamber (202a) of the vibrating mesh nebulizer (202).
6. The drug mixing system (10) according to any one of items 1 to 5, wherein the liquid-releasing means (155, 255) is provided as a part, optionally as an integral part, of the receiving device (200) or of the adapter (100).
7. The drug mixing system (10) according to any one of items 1 to 6, wherein the adapter (100) and the receiving device (200) are respectively equipped with connection means (160) and counter-connection means (260) for connecting the drug mixing system (10) with the receiving device (200).
8. The drug mixing system (10) according to item 7, wherein the connection means (160) and counter-connection means (260) are adapted in such a way that, upon connecting them, the drug mixing system (10) and the receiving device (200) form a leak-proof connection.
9. The drug mixing system (10) according to items 7 or 8, wherein the adapter (100) is equipped with a male connection means (160m) that is insertable into a female counter-connection means (260f) of the receiving device (200).
10. The drug mixing system (10) according to any one of items 7 to 9, wherein the connection means (160) and counter-connection means (260) are adapted in such a way that, upon connecting them, the liquid-releasing means (155, 255) is actuated.
11. The drug mixing system (10) according to item 10, wherein the connection means (160) and counter-connection means (260) are adapted in such a way:
   that upon connecting the seal-type drug mixing system (10a) and the receiving device (200), the septum-piercing means (230) automatically pierces the septum (131); or
   that upon connecting the valve-type drug mixing system (10b) and the receiving device (200), the switch valve (150) is automatically brought into valve position 2.
12. The drug mixing system (10) according to item 11, wherein upon connecting the valve-type drug mixing system (10b) and the receiving device (200), the switch valve (150) is automatically brought into valve position 2 via physical interaction of the connection means (160) and/or the counter-connection means (260) with the switch-valve operating means (156) of the adapter (100b).
13. The drug mixing system (10b) according to item 12, wherein the switch-valve operating means (156) is a part of the adapter (100b) of the valve-type drug mixing system (10b).
14. The drug mixing system (10b) according to item 13, wherein the switch-valve operating means (156) is provided in the form of a protrusion (156a), such as a push-button, jutting out from a male connection means (160m) of the adapter (100b), wherein said male connection means (160m) is insertable into a female counter-connection means (260f) of the receiving device (200), and wherein upon inserting the male connection means (160m) into the female counter-connection means (260f) the protruding switch-valve operating means (156a) is pressed into the male connection means (160m), thereby bringing the switch valve (150) into valve position 2 and expelling, or transferring, the dissolved or diluted drug formulation (110b) from the vial (110) via the outlet port (130) into the receiving device (200) using the overpressure.
15. The drug mixing system (10b) according to item 14, wherein the protruding switch-valve operating means (156a) of the adapter (100b) and/or the female counter-connection means (260f) of the receiving device (200) are provided with angled, or sloped, surfaces to facilitate a controlled, steady movement of the protruding switch-valve operating means (156a) when being pressed into the male connection means (160m).
16. The drug mixing system (10b) according to item 14 or 15, wherein the protruding switch-valve operating means (156a) of the adapter (100b) is covered by a protective wall, or protective cover, that is adapted and positioned such as to prevent premature and/or unintentional movements of the protruding switch-valve operating means (156a) prior to connecting the drug mixing system (10) with the receiving device (200).
17. The drug mixing system (10b) according to item 14 or 16, wherein the receiving device (200) is equipped with a valve-operating pin (256), and wherein upon inserting the male connection means (160m) of the adapter (100b) into the female counter-connection means (260f) of the receiving device (200), the protruding switch-valve operating means (156a) is pressed into the male connection means (160m) by said valve-operating pin (256), thereby bringing the switch valve (150) into valve position 2 and expelling, or transferring, the dissolved or diluted drug formulation (110b) from the vial (110) via the outlet port (130) into the receiving device (200) using the overpressure.
18. The drug mixing system (10) according to any one of items 3 to 11, wherein for a seal-type drug mixing system (10a), the liquid-releasing means (155, 255) is provided in the form of a septum-piercing means (230), and wherein said septum-piercing means (230) is provided as a part, optionally as an integral part, of the receiving device (200).
19. The drug mixing system (10) according to item 18, wherein the septum-piercing means (230) is positioned in the centre of a spoke-wheel shaped part (220) positioned in the receiving device's inlet opening (210) through which the dissolved or diluted drug formulation (110b) is received.
20. The drug mixing system (10) according to item 19, wherein the spoke-wheel shaped part (220) is shaped and/or positioned in the receiving device's inlet opening (210) in such a way that the piercing tip (231) of the septum-piercing means (230) is not jutting out, or protruding, from the surface of the outer casing (240) of the receiving device (200).
21. The drug mixing system (10) according to any one of items 18 to 20, wherein the septum-piercing means (230) is provided in the form of a septum-piercing cannula (230a), or hollow needle, or a so-called ‘spike’.
22. The drug mixing system (10) according to any one of items 1 to 21, wherein the piston locking means (126) is provided in the form of an engagement hook (126a) or a plurality thereof (126a, 126a′, 126a″ etc.), a ratchet mechanism, or a similar snap-fit locking mechanism (126c), or a plurality thereof (126c, 126c′, 126c″ etc).
23. The drug mixing system (10) according to any one of items 1 to 22, wherein the adapter (100) is further equipped with a stabilising wall (170) which externally surrounds, or encases, at least parts of the reservoir's housing (124), wherein the stabilising wall (170) is dimensionally stable so as to support and stabilise the reservoir (120) within the adapter (100) while pressing the piston (125) into the housing (124) and generating the overpressure.
24. The drug mixing system (10) according to item 23, wherein the stabilising wall (170) also externally surrounds, or encases, at least parts of the vial (110).
25. The drug mixing system (10) according to items 23 or 24, wherein at least parts of the stabilising wall (170) are provided as a viewing window (171), for instance, by the stabilising wall (170) being formed from a see-through wall material and/or by providing cut-outs in a wall material.
26. The drug mixing system (10) according to any one of items 1 to 25, wherein the piston locking means (126) is formed as an integral part of the stabilising wall (170).
27. The drug mixing system (10) according to any one of items 1 to 26, wherein the adapter (100) is further equipped with a piston guiding means (127) that enables a controlled, steady movement of the piston (125) within the housing (124) while pressing the piston (125) into the housing (124) and generating the overpressure.
28. The drug mixing system (10) according to item 27, wherein the piston guiding means (127) is provided:
   in the form of a piston guiding groove (127a) in the stabilising wall (170), and a corresponding protrusion (127b) on the piston (125) which slides down the groove (127a) while pressing the piston (125) into the housing (124) and generating the overpressure; or
   in the form of a piston guide rail (127aa) protruding from an inner surface of the stabilising wall (170), and a corresponding notch (127bb) on the piston (125), such as an I-beam shaped guide rail and a corresponding ‘double-T’ notch, which slides down the piston guide rail (127aa) while pressing the piston (125) into the housing (124) and generating the overpressure.
29. The drug mixing system (10) according to any one of items 1 to 28, wherein the vial-mounting opening (111) is equipped with a vial fixation means (114) for unreleasably connecting the vial (110) to the vial-mounting opening (111).
30. The drug mixing system (10) according to any one of items 1 to 29, wherein the reservoir-mounting opening (121) is equipped with a reservoir fixation means (128) for unreleasably connecting the reservoir (120) to the reservoir-mounting opening (121).
31. The drug mixing system (10) according to any one of items 2 to 30, wherein the drug mixing system (10) is a valve-type drug mixing system (10b) with a liquid-retaining means (145) in the form of a switch valve (150), and wherein the switch valve (150) is a three-way switch valve having three openings to selectively enable fluid connection between the vial-mounting opening (111), the reservoir-mounting opening (121), and the outlet port (130).
32. The drug mixing system (10) according to any one of items 2 to 31, wherein the drug mixing system (10) is a valve-type drug mixing system (10b) with a liquid-retaining means (145) in the form of a switch valve (150), and wherein the switch valve (150) is rotatable or shiftable.
33. The drug mixing system (10) according to any one of items 2 to 32, wherein the drug mixing system (10) is a valve-type drug mixing system (10b) with a liquid-retaining means (145) in the form of a switch valve (150), and wherein the switch valve (150) is adapted to switch to a third position, valve position 3, blocking any fluid connection between the vial-mounting opening (111), the reservoir-mounting opening (121), and the outlet port (130).
34. The drug mixing system (10) according to any one of items 1 to 33, wherein the reservoir (120) is provided in the form of a syringe (120c), optionally a syringe equipped with a LUER-slip without a thread (LUER slip) or with a LUER-lock with a thread (LUER lock); or wherein the reservoir (120) is a pharmaceutical cartridge (120b).
35. The drug mixing system (10) according to any one of items 1 to 34, wherein the vial (110) is sealed with a pierceable vial lid (112), and wherein the adapter (100) comprises a respective vial-piercing cannula (113) adapted to pierce the vial lid (112) and access the drug formulation (110a) in the vial (110) during use of the drug mixing system (10).
36. The drug mixing system (10) according to item 35, wherein the reservoir (120) is also sealed with a reservoir lid (122) adapted to open during use of the drug mixing system (10) so as to access the liquid in the reservoir (120).
37. The drug mixing system (10) according to item 35 or 36, wherein the reservoir (120) is sealed with a pierceable reservoir lid (122), and wherein the adapter (100) further comprises a respective reservoir-piercing cannula (123) adapted to puncture the reservoir lid (122) and access the liquid in the reservoir (120) during use of the drug mixing system (10).
38. The drug mixing system (10) according to any one of items 35 to 37, wherein the adapter (100) comprises a first adapter subunit (101), and a second adapter subunit (102), wherein the first and second adapter subunits (101, 102) are connected with one another and engageable in such a way that the adapter (100) has:
   a resting position in which the vial-piercing cannula (113) does not yet pierce the vial lid (112); or, as the case may be, the vial-and reservoir-piercing cannulae (113, 123) do not yet pierce the respective vial-and reservoir lids (112, 122); and
   an activated position in which, during use of the drug mixing system (10), the first and the second adapter subunits (101, 102) are shifted towards each other and thereby brought into a position in which the vial cannula (113) pierces the vial lid (112) thereby accessing the drug formulation (110a) in the vial (110); or, as the case may be, the vial-and reservoir-piercing cannulae (113, 123) pierce the respective vial-and reservoir lids (112, 122), thereby accessing the drug formulation (110a) in the vial (110) and the liquid in the reservoir (120).
39. The drug mixing system (10) according to item 38, wherein the adapter (100) is provided to the user in pre-assembled form, for instance, wherein the first and second adapter subunits (101, 102) of the adapter (100) are pre-assembled.
40. The drug mixing system (10) according to items 38 or 39, wherein, prior to use (such as for shipping, delivery, and storage), the adapter (100) is provided in its resting position.
41. The drug mixing system (10) according to any one of items 38 to 40, wherein, in the resting position, the first and second adapter subunits (101, 102) are unreleasably connected.
42. The drug mixing system (10) according to items 38 to 41, wherein, in the activated position, the first and second adapter subunits (101, 102) are unreleasably connected.
43. The drug mixing system (10) according to any one of items 38 to 42, wherein the first and/or the second adapter subunit (101, 102) comprise at least one adapter subunit locking means (103) for the locking engagement of the first adapter subunit (101) with the second adapter subunit (102).
44. The drug mixing system (10) according to item 43, wherein the adapter subunit locking means (103) is adapted to unreleasably lock the adapter (100) in the resting position or in the activated position.
45. The drug mixing system (10) according to items 43 or 44, wherein the adapter subunit locking means (103) is provided in the form of one or more subunit locking hooks (103a) and corresponding subunit locking grooves (103b) positioned inside the first and second adapter subunits (101, 102).
46. The drug mixing system (10) according to items 43 to 45, wherein the first and/or the second adapter subunit (101, 102) comprise at least two adapter subunit locking means (103) for the engagement of the first adapter subunit (101) with the second adapter subunit (102).
47. The drug mixing system (10) according to item 45, wherein one of the at least two adapter subunit locking means (103) is engaged in the resting position, and the other one is engaged in the activated position.
48. The drug mixing system (10) according to items 38 to 47, wherein the first adapter subunit (101) comprises at least a vial-mounting opening (111) adapted to hold the vial (110), a reservoir-mounting opening (121) adapted to hold the reservoir (120), and optionally the stabilising wall (170) which externally surrounds, or encases, at least parts of the reservoir's housing (124).
49. The drug mixing system (10) according to any one of items 38 to 48, wherein the stabilising wall (170) is formed as an integral part of the first adapter subunit (101).
50. The drug mixing system (10) according to any one of items 38 to 49, wherein the second adapter subunit (102) comprises at least the switch valve (150) and the outlet port (130).
51. The drug mixing system (10) according to any one of items 38 to 50, wherein the adapter (100) comprises adapter subunit guiding means (104) for guiding the movement of the first adapter subunit (101) and the second adapter subunit (102) when shifting them towards each other from the resting position into the activated position.
52. The drug mixing system (10) according to any one of items 38 to 51, wherein the adapter subunit guiding means (104) is provided in the form of at least one subunit guiding recess (104a) in the first adapter subunit (101) and at least one corresponding subunit guiding protrusion (104b) fitting the recess (104a) in the second adapter subunit (102); or vice versa.
53. The drug mixing system (10) according to any one of items 38 to 52, wherein the first and second adapter subunits (101, 102) are adapted such that they can be shifted towards each other single-handedly.
54. The drug mixing system (10) according to any one of items 38 to 53, wherein the adapter (100), preferably the second adapter subunit (102), comprises at least one dedicated site for resting one or more finger(s) (105, 105′) when bringing the adapter (100) from the resting position into the activated position.
55. The drug mixing system (10) according to any one of items 1 to 54, wherein the vial (110) exhibits a longitudinal central axis ‘A’, and wherein said longitudinal central axis ‘A’ is positioned vertical, or essentially vertical, during use.
56. The drug mixing system (10) according to any one of items 5 to 55, wherein the vial (110) exhibits a longitudinal central axis ‘A’ and the receiving device (200) exhibits a longitudinal central axis ‘C’, and wherein the vial's longitudinal central axis ‘A’ is oriented perpendicular to the receiving device's longitudinal central axis ‘C’ when the drug mixing system (10) is connected to the receiving device (200).
57. The drug mixing system (10) according to any one of items 1 to 56, wherein the vial (110) exhibits a longitudinal central axis ‘A’, and the reservoir (120) exhibits a longitudinal central axis ‘B’, and wherein the vial-and reservoir mounting openings (111, 121) are arranged to hold the vial (110) and the reservoir (120) in a position in which longitudinal central axes ‘A’ and ‘B’ are positioned at an angle below 75°, preferably below 60°, more preferably below 50°, or even below 20°, such as 0°.
58. The drug mixing system (10) according to item 57, wherein the longitudinal central axes ‘A’ and ‘B’ are positioned at an angle between 20° and 0°, preferably between 10° and 0°, more preferably between 5° and 0°, such as 0°; or wherein the longitudinal central axes ‘A’ and ‘B’ are positioned at an angle between 70° and 20°, or between 60° and 30°, or between 50° and 40°, such as 45°.
59. The drug mixing system (10) according to any item 57 or 58, wherein the vial-and reservoir mounting openings (111, 121) of the adapter (100) are arranged in such a way that at least the vial's longitudinal central axis ‘A’, optionally both the vial's and the reservoir's longitudinal central axes ‘A’ and ‘B’, is/are positioned vertical, or essentially vertical, during use.
60. The drug mixing system (10) according to any one of items 1 to 59, wherein the adapter (100), and optionally the drug mixing system (10), is sterilizable.
61. The drug mixing system (10) according to item 60, wherein the adapter (100), and optionally the drug mixing system (10), is sterilizable in packaged state.
62. The drug mixing system (10) according to item 60 or 61, wherein the adapter (100), and optionally the drug mixing system (10), is sterilizable with ethylene oxide and/or gamma irradiation.
63. The drug mixing system (10) according to any one of items 1 to 62, wherein the adapter (100), and optionally the drug mixing system (10), is stored and delivered in a pre-packaged, sterilized state.
64. The drug mixing system (10) according to any one of items 1 to 63, wherein the vial (110), and the reservoir (120) are pre-assembled within the respective the vial-and reservoir mounting openings (111, 121) of the adapter (100) of the drug mixing system (10).
65. The drug mixing system (10) according to any one of items 2 to 64, wherein the drug mixing system (10) is a valve-type drug mixing system (10b) with a liquid-retaining means (145) in the form of a switch valve (150), and wherein the adapter (100), and optionally the drug mixing system (10), is stored and delivered in a pre-packaged state with the switch valve (150) of the adapter (100) being in valve position 1.
66. The drug mixing system (10) according to any one of items 1 to 65, wherein the drug formulation (110a) in the vial (110) is provided in powder form, such as a powder for reconstitution, or as a liquid.
67. The drug mixing system (10) according to any one of items 1 to 66, wherein the drug formulation (110a) in the vial (110) is provided in a lyophilized form.
68. The drug mixing system (10) according to any one of items 1 to 67, wherein the drug formulation (110a) in the vial (110) is provided as a liquid, and wherein the liquid is a concentrate and/or a liquid solubilisate of the drug.
69. The drug mixing system (10) according to any one of items 1 to 68, wherein the drug formulation (110a) in the vial (110) comprises at least one drug, optionally a combination of drugs.
70. The drug mixing system (10) according to any one of items 1 to 69, wherein the liquid in the reservoir (120) is a pharmaceutically acceptable liquid.
71. The drug mixing system (10) according to item 70, wherein the pharmaceutically acceptable liquid is an injectable liquid and/or an inhalable liquid, such as water for injection (aqua ad injectabilia).
72. The drug mixing system (10) according to any one of items 1 to 71, the drug mixing system being intended for single-use.
73. An adapter (100) for the drug mixing system (10) according to any one of items 1 to 72.
74. The adapter (100) according to item 73, the adapter (100) is equipped with a piston locking means (126) adapted to lock the position of the piston (125) once a predetermined volume of the liquid has been transferred from the reservoir (120) into the vial (110) by means of pressing the piston (125) into the housing (124), thereby creating and storing an overpressure.
75. The adapter (100) according to item 73 or 74, wherein the adapter (100) is equipped with a stabilising wall (170) which externally surrounds, or encases, at least parts of the reservoir housing (124), said stabilising wall (170) being dimensionally stable so as to support and stabilise the reservoir (120) within the adapter (100) while pressing the piston (125) into the housing (124) and generating the overpressure.
76. A method of preparing a dissolved or diluted drug formulation (110b), optionally a drug solution, from a drug formulation (110a) stored in a vial (110), the method comprising the following subsequent steps:
   a) providing a drug mixing system (10) according to any one of items 1 to 72;
   b) with the liquid-retaining means (145) blocking at least the flow of dissolved or diluted drug formulation (110b) through the outlet port (130), transferring a predetermined volume of the liquid from the reservoir (120) into the vial (110) by means of pressing the piston (125) into the housing (124), thereby creating an overpressure and a mixture of the drug formulation (110a) and the liquid;
   c) locking the piston (125) in place with the piston locking means (126) once the predetermined volume of the liquid has been transferred from the reservoir (120) into the vial (110), thereby storing the overpressure created in step b;
   d) allowing the mixture of the drug formulation (110a) and the liquid obtained from steps b) and c) to get dissolved or diluted to the desired extent; optionally aiding this dissolution or dilution process by shaking the drug mixing system (10);
   e) actuating the liquid-releasing means (155, 255), thereby expelling, or transferring, the dissolved or diluted drug formulation (110b) from the vial (110) through the outlet port (130) using the overpressure generated and stored in steps b) and c) by pressing the piston (125) into the housing (124) and locking the piston (125) in place.
77. The method of preparing a dissolved or diluted drug formulation (110b), optionally a drug solution, from a drug formulation (110a) stored in a vial (110) according to item 76, the method comprising the following subsequent steps:
   a) providing a valve-type drug mixing system (10b) according to any one of the applicable items 2 to 72, with the liquid-retaining means (145) being provided in the form of a switch valve (150), and the internal adapter channel (140) and the switch valve (150) being adapted to at least allow selective fluid connection between the vial (110) and the reservoir (120) while blocking fluid connection to the outlet port (130) when in valve position 1, and between the vial (110) and the outlet port (130) while blocking fluid connection to the reservoir (120) when in valve position 2;
   b) with the switch valve (150) being in valve position 1, or being brought to valve position 1, transferring a predetermined volume of the liquid from the reservoir (120) into the vial (110) by means of pressing the piston (125) into the housing (124), thereby creating an overpressure and a mixture of the drug formulation (110a) and the liquid;
   c) locking the piston (125) in place with the piston locking means (126) once the predetermined volume of the liquid has been transferred from the reservoir (120) into the vial (110), thereby storing the overpressure created in step b;
   d) allowing the mixture of the drug formulation (110a) and the liquid obtained from steps b) and c) to get dissolved or diluted to the desired extent; optionally aiding this dissolution or dilution process by shaking the drug mixing system (10);
   e) bringing the switch valve (150) into valve position 2, thereby expelling, or transferring, the dissolved or diluted drug formulation (110b) is from the vial (110) through the outlet port (130) using the overpressure generated and stored in steps b) and c) by pressing the piston (125) into the housing (124) and locking the piston (125) in place.
78. The method of preparing a dissolved or diluted drug formulation (110b), optionally a drug solution, solution from a drug formulation (110a) stored in a vial (110) according to item 76, the method comprising the following subsequent steps:
a) providing a seal-type drug mixing system (10a) according to any one of the applicable items 2 to 72, with the liquid-retaining means (145) being provided in the form of a septum (131) covering the outlet port (130), and the liquid-releasing means (155, 255) in the form of a septum-piercing means (230);
b) with the septum (131) blocking at least the flow of dissolved or diluted drug formulation (110b) through the outlet port (130), transferring a predetermined volume of the liquid from the reservoir (120) into the vial (110) by means of pressing the piston (125) into the housing (124), thereby creating an overpressure and a mixture of the drug formulation (110a) and the liquid;
c) locking the piston (125) in place with the piston locking means (126) once the predetermined volume of the liquid has been transferred from the reservoir (120) into the vial (110), thereby storing the overpressure created in step b;
d) allowing the mixture of the drug formulation (110a) and the liquid obtained from steps b) and c) to get dissolved or diluted to the desired extent; optionally aiding this dissolution or dilution process by shaking the drug mixing system (10);
e) actuating the liquid-releasing means (155, 255) by piercing the septum (131) with the septum-piercing means (230), thereby expelling, or transferring, the dissolved or diluted drug formulation (110b) from the vial (110) through the outlet port (130) using the overpressure generated and stored in steps b) and c) by pressing the piston (125) into the housing (124) and locking the piston (125) in place.
79. The method according to any one of items 76 to 78, wherein a drug mixing system (10) according to any one of items 38 to 71 is provided in step a, and wherein the drug mixing system (10) is activated by bringing the adapter (100) from the resting position into the activated position.
80. The method according to item 79, wherein the adapter (100) is brought from the resting position into the activated position by shifting the first and second adapter subunits (101, 102) of the adapter (100) towards each other.
81. The method according to item 79 or 80, wherein the first and second adapter subunits (101, 102) of the adapter (100) can be shifted towards each other single-handedly.
82. The method according to any one of items 76 to 81, wherein the dissolved or diluted drug formulation (110b) is transferred from the vial (110) via the outlet port (130) into a receiving device (200) for receiving the dissolved or diluted drug formulation (110b) from the vial (110).
83. The method according to any one of items 76 to 82, wherein the adapter (100) according to any one of the items 73 to 75 is provided separately from a vial (110) housing a drug formulation, and/or a reservoir (120) for housing a liquid for dissolution or dilution of the drug formulation, and wherein the vial (110) and/or the reservoir (120) are then coupled to the respective vial-and reservoir mounting openings (111, 121) of the adapter (100) so as to provide the drug mixing system (10) of method step a).

1. A drug mixing system (10) for dissolving or diluting a drug formulation (110a) stored in a vial (110) prior to use, the drug mixing system (10) comprising an adapter (100) adapted to hold the vial (110) housing the drug formulation (110a), and a reservoir (120) for housing a liquid (120a) for dissolution or dilution of the drug formulation (110a), the reservoir (120) comprising a housing (124) and a piston (125) sealingly movable within the housing (124),
wherein the adapter (100) comprises:
a vial mounting opening (111) adapted to hold the vial (110),
a reservoir mounting opening (121) adapted to hold the reservoir (120),
an outlet port (130),
an internal adapter channel (140) fluidly connecting the vial-and reservoir mounting openings (111, 121), and the outlet port (130), and
a liquid-retaining means (145) adapted to block at least a flow of dissolved or diluted drug formulation (110b) through the outlet port (130),
characterized in that:
the adapter (100) is equipped with a piston locking means (126) adapted to lock the position of the piston (125) once a predetermined volume of the liquid has been transferred from the reservoir (120) into the vial (110) by means of pressing the piston (125) into the housing (124), thereby creating and storing an overpressure,
the drug mixing system (10) is adapted to expel, or transfer, the dissolved or diluted drug formulation (110b) from the vial (110) through the outlet port (130) upon actuation of a liquid-releasing means (155, 255) using the overpressure generated by pressing the piston (125) into the housing (124).

2. The drug mixing system (10) according to claim 1, wherein
a) for a seal-type drug mixing system (10a), the liquid-retaining means (145) is provided in the form of a septum (131) covering the outlet port (130), and the liquid-releasing means (155, 255) is provided in the form of a septum-piercing means (230) so that, upon actuation, the septum (131) is pierced by the septum-piercing means (230) and the dissolved or diluted drug formulation (110b) is expelled, or transferred, from the vial (110) through the outlet port (130) using the overpressure generated by pressing the piston (125) into the housing (124); or
b) for a valve-type drug mixing system (10b), the liquid-retaining means (145) is provided in the form of a switch valve (150), optionally a rotatable or a shiftable switch valve (150r, 150s), wherein the internal adapter channel (140) and the switch valve (150) are adapted to at least allow selective fluid connection between the vial (110) and the reservoir (120) while blocking fluid connection to the outlet port (130) when in valve position 1, and between the vial (110) and the outlet port (130) while blocking fluid connection to the reservoir (120) when in valve position 2; and
the liquid-releasing means (155, 255) is provided in the form of a switch-valve operating means (156) adapted to, upon actuation, bring the switch valve (150) into valve position 2, so that, upon the switch valve (150) being brought into said valve position 2, the dissolved or diluted drug formulation (110b) is expelled, or transferred, from the vial (110) through the outlet port (130) using the over-pressure generated by pressing the piston (125) into the housing (124).

3. The drug mixing system (10) according to claims 1 or 2, wherein the dissolved or diluted drug formulation (110b) is transferred from the vial (110) via the outlet port (130) into a receiving device (200) for receiving the dissolved or diluted drug formulation (110b) from the vial (110); optionally a nebulizer (201); further optionally a vibrating mesh nebulizer (202).

4. The drug mixing system (10) according to any one of claims 1 to 3, wherein the adapter (100) and the receiving device (200) are respectively equipped with connection means (160) and counter-connection means (260) for connecting the drug mixing system (10) with the receiving device (200), and
wherein preferably the connection means (160) and counter-connection means (260) are adapted in such a way that, upon connecting them, the liquid-releasing means (155, 255) is actuated.

5. The drug mixing system (10) according to claim 4, wherein the connection means (160) and counter-connection means (260) are adapted in such a way:
that upon connecting the seal-type drug mixing system (10a) and the receiving device (200), the septum-piercing means (230) automatically pierces the septum (131); or
that upon connecting the valve-type drug mixing system (10b) and the receiving device (200), the switch valve (150) is automatically brought into valve position 2; optionally via physical interaction of the connection means (160) and/or the counter-connection means (260) with the switch-valve operating means (156) of the adapter (100b).

6. The drug mixing system (10) according to claim 5, wherein the switch-valve operating means (156) is a part of the adapter (100b) of the valve-type drug mixing system (10b), and
wherein the switch-valve operating means (156) is provided in the form of a protrusion (156a), such as a push-button, jutting out from a male connection means (160m) of the adapter (100b),
wherein said male connection means (160m) is insertable into a female counter-connection means (260f) of the receiving device (200), and
wherein upon inserting the male connection means (160m) into the female counter-connection means (260f) the protruding switch-valve operating means (156a) is pressed into the male connection means (160m), thereby bringing the switch valve (150) into valve position 2 and expelling, or transferring, the dissolved or diluted drug formulation (110b) from the vial (110) via the outlet port (130) into the receiving device (200) using the overpressure.

7. The drug mixing system (10) according to any one of claims 3 to 5, wherein for a seal-type drug mixing system (10a), the liquid-releasing means (155, 255) is provided in the form of a septum-piercing means (230), and wherein the liquid-releasing means (155, 255) is provided as apart of the receiving device (200), and wherein optionally the septum-piercing means (230) is positioned in the centre of a spoke-wheel shaped part (220) positioned in the receiving device's inlet opening (210) through which the dissolved or diluted drug formulation (110b) is received.

8. The drug mixing system (10) according to any one of claims 1 to 7, wherein the piston locking means (126) is provided in the form of an engagement hook (126a) or a plurality thereof (126a, 126a′, 126a″ etc.), a ratchet mechanism, or a similar snap-fit locking mechanism (126c), or a plurality thereof (126c, 126c′, 126c″ etc).

9. The drug mixing system (10) according to any one of claims 1 to 8, wherein the adapter (100) is further equipped with a stabilising wall (170) which externally surrounds, or encases, at least parts of the reservoir's housing (124), wherein the stabilising wall (170) is dimensionally stable so as to support and stabilise the reservoir (120) within the adapter (100) while pressing the piston (125) into the housing (124) and generating the overpressure.

10. The drug mixing system (10) according to any one of claims 1 to 9, wherein the adapter (100) is further equipped with a piston guiding means (127) that enables a controlled, steady movement of the piston (125) within the housing (124) while pressing the piston (125) into the housing (124) and generating the overpressure.

11. The drug mixing system (10) according to any one of claims 2 to 10, wherein the drug mixing system (10) is a valve-type drug mixing system (10b) with a liquid-retaining means (145) in the form of a switch valve (150), and wherein the switch valve (150) is a three-way switch valve having three openings to selectively enable fluid connection between the vial-mounting opening (111), the reservoir-mounting opening (121), and the outlet port (130).

12. The drug mixing system (10) according to any one of claims 1 to 11, wherein the vial (110) exhibits a longitudinal central axis ‘A’, and wherein said longitudinal central axis ‘A’ is positioned vertical, or essentially vertical, during use.

13. The drug mixing system (10) according to any one of claims 1 to 12, wherein the vial (110), and the reservoir (120) are pre-assembled within the respective the vial-and reservoir mounting openings (111, 121) of the adapter (100) of the drug mixing system (10).

14. An adapter (100) for the drug mixing system (10) according to any one of claims 1 to 13.

15. A method of preparing a dissolved or diluted drug formulation (110b), optionally a drug solution, from a drug formulation (110a) stored in a vial (110), the method comprising the following subsequent steps:
a) providing a drug mixing system (10) according to any one of claims 1 to 13;
b) with the liquid-retaining means (145) blocking at least the flow of dissolved or diluted drug formulation (110b) through the outlet port (130), transferring a predetermined volume of the liquid from the reservoir (120) into the vial (110) by means of pressing the piston (125) into the housing (124), thereby creating an overpressure and a mixture of the drug formulation (110a) and the liquid;
c) locking the piston (125) in place with the piston locking means (126) once the predetermined volume of the liquid has been transferred from the reservoir (120) into the vial (110), thereby storing the overpressure created in step b;
d) allowing the mixture of the drug formulation (110a) and the liquid obtained from steps b) and c) to get dissolved or diluted to the desired extent; optionally aiding this dissolution or dilution process by shaking the drug mixing system (10);
e) actuating the liquid-releasing means (155, 255), thereby expelling, or transferring, the dissolved or diluted drug formulation (110b) from the vial (110) through the outlet port (130) using the overpressure generated and stored in steps b) and c) by pressing the piston (125) into the housing (124) and locking the piston (125) in place.
